(19) United States
(12) Patent Application Publication
(10) Pub. No.: US 2011/0086063 A1
(43) Pub. Date: Apr. 14, 2011

(54) VACCINES FOR PREVENTION AND TREATMENT OF ADDICTION

(75) Inventors: Ronald G. Crystal, New York, NY (US); Philip L. Leopold, Basking Ridge, NJ (US); Stefan Worgall, New York, NY (US); Julie L. Boyer, New York, NY (US)

(73) Assignee: CORNELL UNIVERSITY, Ithaca, NY (US)

(21) Appl. No.: 12/995,851

(22) PCT Filed: Jun. 4, 2009
(86) PCT No.: PCT/US2009/046248

§ 371 Date: Dec. 2, 2010

Related U.S. Application Data
(60) Provisional application No. 61/058,698, filed on Jun. 4, 2008.

(51) Int. Cl. A61K 39/235; A61K 31/7088; A61P 25/36; A61P 25/30; A61P 25/34; C12N 7/00; C12N 15/63

(52) U.S. Cl. 424/199.1; 424/233.1; 514/44 R; 435/235.1; 435/320.1

(57) ABSTRACT

The invention provides an adenovirus-antigen conjugate comprising an adenovirus with a coat protein and an antigen of an addictive drug conjugated to the coat protein of the adenovirus. The invention also provides an adenoviral vector comprising a nucleic acid sequence which encodes an antibody directed against the addictive drug. The invention further provides a method of inducing an immune response against an addictive drug or reducing the effect of an addictive drug in a human by ad-ministering to the human the aforementioned adenovirus-antigen conjugate or antibody encoding adenoviral vector.

### CROSS-REFERENCE TO RELATED APPLICATIONS

This patent application claims the benefit of U.S. Provisional Patent Application No. 61/058,698, filed Jun. 4, 2008, which is incorporated by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,039 Byte ASCII (Text) file named “704918_ST25.txt” created on Jun. 3, 2009.

### BACKGROUND OF THE INVENTION

Addiction to drugs is a major problem worldwide. Although a variety of strategies are in use to prevent and treat drug addiction, major economic and social costs are associated with drug addiction.

The most widely used addictive drug in the world is tobacco, of which the principal addictive component is nicotine. Worldwide, there are >1 billion tobacco smokers, with an estimated 4.9 million tobacco-related deaths per year. The National Center for Health Statistics estimates that 21% of adults in the United States smoke cigarettes (Center for Disease Control and Prevention, 2004 data). Each puff of cigarette smoke contains >4000 chemicals, including a dose of >10¹⁵ oxidant molecules (Church and Prior, Environ. Health. Perspect., 64: 111-126 (1985)). Cigarette smoke causes inflammation and is directly toxic to differentiated airway cells and causes lung cancer, emphysema, and chronic bronchitis. Systemically, smoking has been associated with an increased risk of coronary heart disease, strokes, and a variety of other neoplasms (Fiore et al., Respir. Care, 45: 1196-1199 (2000)). According to the report of the Surgeon General (2004), smoking-related health care is thought to cost in excess of $150 billion annually in the United States. Upon inhalation of cigarette smoke, nicotine passes into the bloodstream, and within seconds, crosses the blood brain barrier where it stimulates brain cells to perceive immediate reward and pleasure. This response is central to the addictive properties of nicotine and is associated with a high relapse rate to smokers who attempt to quit. Whereas the majority of smokers report they want to quit, less than 5% who attempt to do so are able to stay tobacco-free.

In addition to nicotine, strategies to treat and prevent addiction to other drugs are also needed. Examples of such drugs include opioids and morphine derivatives (e.g., cocaine, fentanyl and its analogs, heroin, morphine, opium, oxycodone, and hydrocodone), dissociative anesthetics (e.g., ketamine and PCP and analogs), depressants (e.g., barbiturates, benzodiazepines, flunitrazepam, QHB, and methaquakme), cannabinoids (e.g., hashish and marijuana), hallucinogens (e.g., LSD, mescaline, and psilocybin), stimulants (e.g., amphetamine, cocaine, MDMA, methamphetamine, methylphenidate, and nicotine), and a variety of other drugs such as prescription medications (e.g., opioid pain relievers), anabolic steroids, inhalants, and club drugs.

Despite decades of effort focused upon developing strategies to prevent and treat drug addiction, very little success has been achieved. In the case of nicotine addiction, active behavioral interventions such as individual or group counseling or cognitive therapy alone or in combination with drug therapies such as nicotine replacement therapy (e.g., via chewing gum, transdermal patches, nasal sprays, inhalers, or lozenges), bupropion (ZYBAN™), and varenicline (CHANTIX™), have improved the rates of achieving successful quitting, but the success rates remain only 1.5- to 2.0-fold over placebo, with long term (1 yr) smoking cessation rates of only 5 to 20%. There has been a similar lack of success in the treatment of cocaine addition, and there are no small molecule, monoclonal antibody, or enzyme therapies that have been approved for treatment of cocaine addiction.

Vaccines represent another strategy to prevent and treat drug addiction, and results with vaccines against nicotine and other small molecules such as cocaine and morphine/heroin have been described (Carrera et al., Proc. Natl. Acad. Sci. USA, 98: 1988-1992 (2001); Anton and Leff, Vaccine 24: 3232-3240 (2006); Carrera et al., Nature 379: 727-730 (1995); Hatsukami et al., Clin. Pharmacol. Ther., 78: 456-467 (2005); Maurer et al., Eur. J. Immunol., 35: 2031-2040 (2005)). A major hurdle in the development of effective vaccines is that most addictive drugs, like most small molecules, are poor immunogens. The immunogenicity of addictive drugs can be enhanced by chemically conjugating a drug (or analog thereof) to a larger molecule, such as a protein, and vaccines employing this strategy have been tested in animals and humans (see, e.g., Bonese, et al., Nature 252: 708-710 (1974); Killian, et al., Pharmacol. Biochem. Behay. 9: 347-352 (1978); Carrera et al., Nature 379: 727-730 (1995); Carrera et al., Proc. Nat. Acad. Sci. USA, 98: 1988-1992 (2001); and Carrera et al., Proc. Nat. Acad. Sci. USA, 97: 6202-6206 (2000)). Antibodies directed against certain addictive drugs have also been described (see, e.g., Hardin et al., J Pharmacol Exp Ther 285: 1113-1122 (1998); Proksch et al., J. Pharmacol Exp Ther. 292: 831-837 (2000); and Byrnes-Blake et al., Int Immunopharmacol 1: 329-338 (2001)).

The feasibility of an anti-nicotine vaccine has been demonstrated in principle (see, e.g., Hieda et al., J. Pharm. Exp. Ther. 283: 1076-1081 (1997); Hieda et al., Psychopharm., 143: 150-157 (1999); Hieda et al., Int. J. Immunopharm. 22: 809-819 (2000); Pentel et al., Pharm. Biochem. Behay., 65: 191-198 (2000), Malin et al., Pharm. Biochem. Behay., 68: 87-92 (2001); and International Patent Application Publication WO 99/61054)).

Currently, there are three anti-nicotine vaccines in human clinical trials: (1) TA-NIC (Celtic Pharma, Hamilton, Bermuda), which comprises nicotine linked to recombinant cholera toxin B; (2) NIC002 (formerly CYT002-NicQb) (Cytos Biotechnology, Zurich, Switzerland), which comprises nicotine linked to virus-like particles from the Qb bacteriophage; and (3) NICVAX™ (Nabi Biopharmaceuticals, Boca Raton, Fla.), which comprises nicotine linked to recombinant exoprotein A. Although the results to date suggest that the vaccines are well tolerated, the efficacy of these vaccine strategies is not clear. In particular, the clinical trial data suggest that only the patients with the highest serum titer of anti-nicotine antibodies receive a clinical benefit from the vaccine (see, e.g., Le H J, Clin. Pharmacol. Ther., 78: 453-455 (2005)).

Thus, there is a need for alternative compositions and methods to prevent or treat drug addiction. This invention provides such compositions and methods. This and other advantages of the invention will become apparent from the detailed description provided herein.

### BRIEF SUMMARY OF THE INVENTION

The invention provides a method of inducing an immune response against an addictive drug in a human, which method comprises administering to a human an adenovirus-antigen conjugate comprising an adenovirus with a coat protein and an antigen of an addictive drug conjugated to the coat protein of the adenovirus, whereby the antigen is presented to the immune system of the human to induce an immune response against the addictive drug in the human.

The invention additionally provides a method of reducing the effect of an addictive drug in a human, which method comprises administering to a human an adenoviral vector comprising a nucleic acid sequence which encodes an antibody directed against an addictive drug and which is operably linked to a promoter, whereby the nucleic acid is expressed in the human to produce the antibody and reduce the effect of the addictive drug in the human.

The invention also provides an adenovirus-antigen conjugate comprising an adenovirus with a coat protein and an antigen of an addictive drug conjugated to the coat protein of the adenovirus. Further provided is an adenoviral vector comprising a nucleic acid sequence which encodes an antibody directed against an addictive drug and which is operably linked to a promoter, wherein the nucleic acid can be expressed in a human to produce the antibody. The invention also provides compositions comprising (a) the aforementioned adenovirus-antigen conjugate or the adenoviral vector and (b) a carrier therefor.

### BRIEF DESCRIPTION OF THE DRAWINGS

FIG. 1A is a Western blot of replication-defective human serotype 5 Ad gene transfer vectors conjugated to the nicotine analog, AM3, at various ratios of Ad5:AM3 including 1:1 (lane 2), 1:3 (lane 3), 1:10 (lane 4), 1:30 (lane 5), 1:100 (lane 6), 1:300 (lane 7), and 1:1000 (lane 8) probed with a nicotine-specific antibody. Unconjugated Ad5 is the negative control (lane 1). The positions of the viral hexon, penton base, and fiber proteins are indicated. FIG. 1B is a line graph depicting the time course of anti-AM3 antibody titer induction following immunization of BALB/c mice (n=3/group) with AdAM3 conjugates prepared at the indicated ratios of Ad5:AM3, or as a negative control, an unconjugated Ad5 vector, at a dose of 10¹⁰ pu. At weekly intervals, serum anti-AM3 antibody titers were measured by ELISA. Four and eight weeks (wk) post-immunization, the mice received a boost of the homologous conjugate.

FIG. 2 is a line graph depicting the time course of anti-GNC antibody titer induction following immunization of BALB/c mice (n=3/group) with AdGNC conjugates prepared at the indicated ratios of Ad5:GNC, or as a negative control, an unconjugated AdLacZ vector, at a dose of 10¹⁰ pu. At weekly intervals, serum anti-GNC antibody titers were measured by ELISA. At four weeks (wk) post-immunization, the mice received a boost of the homologous conjugate.

FIG. 3 is a line graph depicting the time course of anti-GNC antibody titer induction following immunization of BALB/c mice (n=3/group) with AdC7GNC conjugates prepared at the indicated ratios of AdC7:GNC, or as a negative control, an unconjugated AdC7 vector, at a dose of 10¹⁰ pu. At the indicated time intervals, serum anti-GNC antibody titers were measured by ELISA.

FIG. 4 is a diagram of an Ad5-based E1-, E3-replication-defective vaccine vector modified to increase primary amines for hapten conjugation, wherein 10 lysine residues (K10) are inserted into the hypervariable regions of the Ad hexon (hex) coding sequence. The base vector can be any Ad serotype and a variable number of lysine residues can be incorporated into the hexon. The vector may comprise a transgene that encodes, e.g., a protein that stimulates B cell activity.

FIG. 5 is a bar graph depicting cocaine and candy self-administration in female rhesus monkeys. The data is presented as a function of cocaine dose available.

### DETAILED DESCRIPTION OF THE INVENTION

The invention is premised, at least in part, on the appreciation that an effective addictive drug vaccine can be generated by conjugating an antigen of an addictive drug, or derivative thereof, to the capsid of an adenovirus. The reason that the adenovirus is an ideal carrier for the antigen of the addictive drug is that the adenovirus avidly interacts with antigen presenting cells (e.g., dendritic cells), and thus acts as an adjuvant to evoke immunity against itself. By coupling the antigen of the addictive drug to one or more of the adenovirus capsid proteins (e.g., hexon, penton base, fiber, protein IX, or other proteins), the immune system treats the antigen of the addictive drug as part of the adenovirus, and generates immunity against the drug.

While not wishing to be bound to any particular theory, it is believed that the addictive drug (or a derivative or analog thereof) becomes highly immunogenic because of the inherent properties of the adenovirus capsid, including its size, binding affinities (both endogenous as well as with genetically engineered enhanced binding affinities), and the ability of the adenovirus to co-deliver immunoactivators as transgenes within its genome. It is further believed that viruses, and in particular, adenoviruses, represent an especially effective form of nanoparticle vaccines due to their size (80-90 nm) and their ability to interact with the cell surface as part of their infection cycle.

The invention provides a method of inducing an immune response against an addictive drug in a human. The method comprises administering to a human an adenovirus-antigen conjugate comprising (a) an adenovirus with a coat protein and (b) an antigen of an addictive drug conjugated to the coat protein of the adenovirus, whereby the antigen is presented to the immune system of the human to induce an immune response against the addictive drug in the human.

Adenovirus (Ad) is a 36 kb double-stranded DNA virus that efficiently transfers DNA in vivo to a variety of different target cell types. The term “adenovirus,” as used herein, includes “adenoviral vectors” as well as “adenoviral particles” or “adenovirus virions” propagated from adenoviral vectors. Thus, the terms “adenovirus,” “adenoviral vectors,” “adenoviral particles,” and “adenovirus virions” are synonymous and can be used interchangeably. In the context of the inventive method, adenoviruses from various origins, subtypes, or mixture of subtypes can be used as the source of the viral genome for the adenoviral vector. While non-human adenovirus (e.g., simian, avian, canine, ovine, or bovine adenoviruses) can be used to generate the adenoviral vector, a human adenovirus preferably is used as the source of the viral genome for the adenoviral vector. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 (i.e., Ad1 through Ad51) are available from the American Type Culture Collection (ATCC, Manassas, Va.). Preferably, in the context of the invention, the adenoviral vector is of human subgroup C, especially serotype 2 or even more desirably serotype 5. However, non-group C adenoviruses can be used in the context of the invention. Preferred adenoviruses used in the construction of non-group C adenoviral vectors include Ad12 (group A), Ad7 and Ad35 (group B), Ad28 and Ad30 (group D), Ad4 (group E), and Ad41 (group F). Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and International Patent Application Publications WO 97/12986 and WO 98/53087.

The adenoviral vector can comprise a mixture of subtypes and thereby be a “chimeric” adenoviral vector. A chimeric adenoviral vector can comprise an adenoviral genome that is derived from two or more (e.g., 2, 3, 4, etc.) different adenovirus serotypes. In the context of the invention, a chimeric adenoviral vector can comprise approximately different or equal amounts of the genome of each of the two or more different adenovirus serotypes.

To circumvent pre-existing anti-adenovirus immunity in humans, vectors based on novel adenovirus serotypes that are not human pathogens have been developed, including the C7 vector, which is based on a chimpanzee adenovirus [Farina et al., J. Virol., 75: 11603-11613 (2001) and Hashimoto et al., Infect. Immun., 73: 6885-6891 (2005)). Therefore, the adenoviral vector also can be based on a non-human primate adenovirus. For example, the adenovirus can be AdC7. Non-human primate serotypes do not circulate in the human population and, consequently, humans do not have pre-existing serum neutralizing antibodies. Even in the presence of pre-existing Ad5 immunity, vaccines based on the chimpanzee-derived serotype AdC7 are effective in generating potent transgene product-specific humoral immune responses against relevant antigens from a variety of pathogens.

The adenoviral vector of the invention can be replication-competent. For example, the adenoviral vector can have a mutation (e.g., a deletion, an insertion, or a substitution) in the adenoviral genome that does not inhibit viral replication in host cells. The adenoviral vector also can be conditionally replication-competent. Preferably, however, the adenoviral vector is replication-deficient in host cells.

By “replication-deficient” or “replication-defective” it is meant that the adenoviral vector requires complementation of one or more regions of the adenoviral genome that are required for replication, as a result of, for example, a deficiency in at least one replication-essential gene function (i.e., such that the adenoviral vector does not replicate in typical host cells, especially those in a human patient that could be infected by the adenoviral vector in the course of the inventive method). A deficiency in a gene, gene function, gene, or genomic region, as used herein, is defined as a mutation or deletion of sufficient genetic material of the viral genome to obliterate or impair the function of the gene (e.g., such that the function of the gene product is reduced by at least about 2-fold, 5-fold, 10-fold, 20-fold, 30-fold, or 50-fold) whose nucleic acid sequence was mutated or deleted in whole or in part. Deletion of an entire gene region often is not required for disruption of a replication-essential gene function. However, for the purpose of providing sufficient space in the adenoviral genome for one or more transgenes, removal of a majority of a gene region may be desirable. While deletion of genetic material is preferred, mutation of genetic material by addition or substitution also is appropriate for disrupting gene function. Replication-essential gene functions are those gene functions that are required for replication (e.g., propagation) and are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA1 and/or VA-RNA-2).

The replication-deficient adenoviral vector desirably requires complementation of at least one replication-essential gene function of one or more regions of the adenoviral genome for viral replication. Preferably, the adenoviral vector requires complementation of at least one gene function of the E1A region, the E1B region, or the E4 region of the adenoviral genome required for viral replication (denoted an E1-deficient or E4-deficient adenoviral vector). Most preferably, the adenoviral vector is deficient in at least one replication-essential gene function (desirably all replication-essential gene functions) of the E1 region and at least one gene function of the nonessential E3 region (e.g., an Xba I deletion of the E3 region) (denoted an E1/E3-deficient adenoviral vector). With respect to the E1 region, the adenoviral vector can be deficient in part or all of the E1A region and/or part or all of the E1B region, e.g., in at least one replication-essential gene function of each of the E1A and E1B regions, thus requiring complementation of the E1A region and the E1B region of the adenoviral genome for replication. The adenoviral vector also can require complementation of the E4 region of the adenoviral genome for replication, such as through a deficiency in one or more replication-essential gene functions of the E4 region.

When the adenoviral vector is deficient in at least one replication-essential gene function in one region of the adenoviral genome (e.g., an E1- or E1/E3-deficient adenoviral vector), the adenoviral vector is referred to as “singly replication-deficient.” A particularly preferred singly replication-deficient adenoviral vector is, for example, a replication-deficient adenoviral vector requiring, at most, complementation of the E1 region of the adenoviral genome, so as to propagate the adenoviral vector (e.g., to form adenoviral vector particles).

The adenoviral vector can be “multiply replication-deficient,” meaning that the adenoviral vector is deficient in one or more replication-essential gene functions in each of two or more regions of the adenoviral genome, and requires complementation of those functions for replication. For example, the aforementioned E1-deficient or E1/E3-deficient adenoviral vector can be further deficient in at least one replication-essential gene function of the E4 region (denoted an E1/E4- or E1/E3/E4-deficient adenoviral vector), and/or the E2 region (denoted an E1/E2- or E1/E2/E3-deficient adenoviral vector), preferably the E2A region (denoted an E1/E2A- or E1/E2A/E3-deficient adenoviral vector).

Desirably, the adenoviral vector requires, at most, complementation of replication-essential gene functions of the E1, E2A, and/or E4 regions of the adenoviral genome for replication (i.e., propagation). However, the adenoviral genome can be modified to disrupt one or more replication-essential gene functions as desired by the practitioner, so long as the adenoviral vector remains deficient and can be propagated using, for example, complementing cells and/or exogenous DNA (e.g., helper adenovirus) encoding the disrupted replication-essential gene functions. In this respect, the adenoviral vector can be deficient in replication-essential gene functions of only the early regions of the adenoviral genome, only the late regions of the adenoviral genome, both the early and late regions of the adenoviral genome, or all adenoviral genes (i.e., a high capacity adenovector (HC-Ad), see Morsy et al., Proc. Natl. Acad. Sci. USA, 95: 965-976 (1998), Chen et al., Proc. Natl. Acad. Sci. USA, 94: 1645-1650 (1997), and Kochanek et al., Hum. Gene Ther., 10: 2451-2459 (1999)). Suitable replication-deficient adenoviral vectors, including singly and multiply replication-deficient adenoviral vectors, are disclosed in U.S. Pat. Nos. 5,837,511, 5,851,806, 5,994,106, 6,127,175, 6,482,616, and 7,195,896; U.S. Patent Application Publications 2001/0043922 A1, 2002/0004040 A1, 2002/0110545 A1, and 2004/0161848 A1; and International Patent Application Publications WO 94/28152, WO 95/02697, WO 95/16772, WO 95/34671, WO 96/22378, WO 97/12986, WO 97/21826, and WO 03/022311.

In addition to modification (e.g., deletion, mutation, or replacement) of adenoviral sequences encoding replication-essential gene functions, the adenoviral genome can contain benign or non-lethal modifications, i.e., modifications which do not render the adenovirus replication-deficient, or, desirably, do not adversely affect viral functioning and/or production of viral proteins, even if such modifications are in regions of the adenoviral genome that otherwise contain replication-essential gene functions. Such modifications commonly result from DNA manipulation or serve to facilitate expression vector construction. For example, it can be advantageous to remove or introduce restriction enzyme sites in the adenoviral genome. Such benign mutations often have no detectable adverse effect on viral functioning.

Replication-deficient adenoviral vectors are typically produced in complementing cell lines that provide gene functions not present in the replication-deficient adenoviral vectors, but required for viral propagation, at appropriate levels in order to generate high titers of viral vector stock. Desirably, the complementing cell line comprises, integrated into the cellular genome, adenoviral nucleic acid sequences which encode gene functions required for adenoviral propagation. The cell line preferably is further characterized in that it contains the complementing genes in a non-overlapping fashion with the adenoviral vector, which minimizes, and practically eliminates, the possibility of the vector genome recombining with the cellular DNA. Accordingly, the presence of replication competent adenoviruses (RCA) is minimized if not avoided in the vector stock, which, therefore, is suitable for certain therapeutic purposes, especially vaccination purposes. The lack of RCA in the vector stock avoids the replication of the adenoviral vector in non-complementing cells. Construction of such a complementing cell lines involve standard molecular biology and cell culture techniques, such as those described by Sambrook et al., Molecular Cloning, a Laboratory Manual, 3^{rd} edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994).

Complementing cell lines for producing the adenoviral vector include, but are not limited to, 293 cells (described in, e.g., Graham et al., J. Gen. Virol., 36, 59-72 (1977)), PER.C6 cells (described in, e.g., International Patent Application Publication WO 97/00326, and U.S. Pat. Nos. 5,994,128 and 6,033,908), and 293-ORF6 cells (described in, e.g., International Patent Application Publication WO 95/34671 and Brough et al., J. Virol., 71: 9206-9213 (1997)). Additional complementing cells are described in, for example, U.S. Pat. Nos. 6,677,156 and 6,682,929, and International Patent Application Publication WO 03/20879. In some instances, the cellular genome need not comprise nucleic acid sequences, the gene products of which complement for all of the deficiencies of a replication-deficient adenoviral vector. One or more replication-essential gene functions lacking in a replication-deficient adenoviral vector can be supplied by a helper virus, e.g., an adenoviral vector that supplies in trans one or more essential gene functions required for replication of the desired adenoviral vector. Helper virus is often engineered to prevent packaging of infectious helper virus. For example, one or more replication-essential gene functions of the E1 region of the adenoviral genome are provided by the complementing cell, while one or more replication-essential gene functions of the E4 region of the adenoviral genome are provided by a helper virus.

If the adenoviral vector is not replication-deficient, ideally the adenoviral vector is manipulated to limit replication of the vector to within a target tissue. The adenoviral vector can be a conditionally-replicating adenoviral vector, which is engineered to replicate under conditions pre-determined by the practitioner. For example, replication-essential gene functions, e.g., gene functions encoded by the adenoviral early regions, can be operably linked to an inducible, repressible, or tissue-specific transcription control sequence, e.g., promoter. In this embodiment, replication requires the presence or absence of specific factors that interact with the transcription control sequence. Conditionally-replicating adenoviral vectors are described further in U.S. Pat. No. 5,998,205.

The coat protein (e.g., hexon, fiber, and penton base) of an adenoviral vector can be manipulated to alter the binding specificity or recognition of a virus for a viral receptor on a potential host cell. For adenovirus, such manipulations can include deletion of regions of the fiber, penton, or hexon, insertions of various native or non-native ligands into portions of the coat protein, and the like. Manipulation of the coat protein can broaden the range of cells infected by the adenoviral vector or enable targeting of the adenoviral vector to a specific cell type.

Any suitable technique for altering native binding to a host cell, such as native binding of the fiber protein to the coxsackievirus and adenovirus receptor (CAR) of a cell, can be employed. For example, differing fiber lengths can be exploited to ablate native binding to cells. This optionally can be accomplished via the addition of a binding sequence to the penton base or fiber knob. This addition of a binding sequence can be done either directly or indirectly via a bispecific or multispecific binding sequence. In an alternative embodiment, the adenoviral fiber protein can be modified to reduce the number of amino acids in the fiber shaft, thereby creating a “short-shafted” fiber (as described in, for example, U.S. Pat. No. 5,962,311). Use of an adenovirus comprising a short-shafted adenoviral fiber gene reduces the level or efficiency of adenoviral fiber binding to its cell-surface receptor and increases adenoviral penton base binding to its cell-surface receptor, thereby increasing the specificity of binding of the adenovirus to a given cell. Alternatively, use of an adenovirus comprising a short-shafted fiber enables targeting of the adenovirus to a desired cell-surface receptor by the introduction of a normative amino acid sequence either into the penton base or the fiber knob.

In yet another embodiment, the nucleic acid residues encoding amino acid residues associated with native substrate binding can be changed, supplemented, or deleted (see, e.g., International Patent Application Publication WO 00/15823, Einfeld et al., J. Virol., 75(23): 11284-11291 (2001), and van Beusechem et al., J. Virol., 76(6): 2753-2762 (2002)) such that the adenoviral vector incorporating the mutated nucleic acid residues (or having the fiber protein encoded thereby) is less able to bind its native substrate. In this respect, the native CAR and integrin binding sites of the adenoviral vector, such as the knob domain of the adenoviral fiber protein and an Arg-Gly-Asp (RGD) sequence located in the adenoviral penton base, respectively, can be removed or disrupted. Any suitable amino acid residue(s) of a fiber protein that mediates or assists in the interaction between the knob and CAR can be mutated or removed, so long as the fiber protein is able to trimerize. Similarly, amino acids can be added to the fiber knob as long as the fiber protein retains the ability to trimerize. Suitable residues include amino acids within the exposed loops of the serotype 5 fiber knob domain, such as, for example, the AB loop, the DE loop, the FG loop, and the HI loop, which are further described in, for example, Roelvink et al., Science, 286: 1568-1571 (1999), and U.S. Pat. No. 6,455,314. Any suitable amino acid residue(s) of a penton base protein that mediates or assists in the interaction between the penton base and integrins can be mutated or removed. Suitable residues include, for example, one or more of the five RGD amino acid sequence motifs located in the hypervariable region of the Ad5 penton base protein (as described, for example, in U.S. Pat. No. 5,731,190). The native integrin binding sites on the penton base protein also can be disrupted by modifying the nucleic acid sequence encoding the native RGD motif such that the native RGD amino acid sequence is conformationally inaccessible for binding to the αv integrin receptor, such as by inserting a DNA sequence into or adjacent to the nucleic acid sequence encoding the adenoviral penton base protein. Preferably, the adenoviral vector comprises a fiber protein and a penton base protein that do not bind to CAR and integrins, respectively. Alternatively, the adenoviral vector comprises fiber protein and a penton base protein that bind to CAR and integrins, respectively, but with less affinity than the corresponding wild-type coat proteins. The adenoviral vector exhibits reduced binding to CAR and integrins if a modified adenoviral fiber protein and penton base protein binds CAR and integrins, respectively, with at least about 5-fold, 10-fold, 20-fold, 30-fold, 50-fold, or 100-fold less affinity than a non-modified adenoviral fiber protein and penton base protein of the same serotype.

The adenoviral vector also can comprise a chimeric coat protein comprising a non-native amino acid sequence that binds a substrate (i.e., a ligand), such as a cellular receptor other than CAR the αv integrin receptor. Such a chimeric coat protein allows an adenoviral vector to bind and, desirably, infect host cells not naturally infected by the corresponding adenovirus that retains the ability to bind native cell surface receptors, thereby further expanding the repertoire of cell types infected by the adenoviral vector. The non-native amino acid sequence of the chimeric adenoviral coat protein allows an adenoviral vector comprising the chimeric coat protein to bind and, desirably, infect host cells not naturally infected by a corresponding adenovirus without the non-native amino acid sequence (i.e., host cells not infected by the corresponding wild-type adenovirus), to bind to host cells naturally infected by the corresponding adenovirus with greater affinity than the corresponding adenovirus without the non-native amino acid sequence, or to bind to particular target cells with greater affinity than non-target cells. A “non-native” amino acid sequence can comprise an amino acid sequence not naturally present in the adenoviral coat protein or an amino acid sequence found in the adenoviral coat but located in a non-native position within the capsid. By “preferentially binds” is meant that the non-native amino acid sequence binds a receptor, such as, for instance, αvβ3 integrin, with at least about 3-fold greater affinity (e.g., at least about 5-fold, 10-fold, 15-fold, 20-fold, 25-fold, 35-fold, 45-fold, or 50-fold greater affinity) than the non-native ligand binds a different receptor, such as, for instance, αvβ1 integrin.

In one embodiment, the adenoviral vector comprises a chimeric coat protein comprising a non-native amino acid sequence that confers to the chimeric coat protein the ability to bind to an immune cell more efficiently than a wild-type adenoviral coat protein. In particular, the adenoviral vector can comprise a chimeric adenoviral fiber protein comprising a non-native amino acid sequence which facilitates uptake of the adenoviral vector by immune cells, preferably antigen presenting cells, such as dendritic cells, monocytes, and macrophages. In a preferred embodiment, the adenoviral vector comprises a chimeric fiber protein comprising an amino acid sequence (e.g., a non-native amino acid sequence) comprising an RGD motif including, but not limited to, CRGDC (SEQ ID NO: 1), CXCRGDCXC (SEQ ID NO: 2), wherein X represents any amino acid, and CDCRGDCFC (SEQ ID NO: 3), which increases transduction efficiency of an adenoviral vector into dendritic cells. The RGD-motif, or any non-native amino acid sequence, can be inserted into the adenoviral fiber knob region, ideally in an exposed loop of the adenoviral knob, such as the HI loop. A non-native amino acid sequence also can be appended to the C-terminus of the adenoviral fiber protein or hexon protein, optionally via a spacer sequence. The spacer sequence can comprise between one and two-hundred amino acids, and can (but need not) have an intended function.

In another embodiment, the adenoviral vector can comprise a chimeric virus coat protein that is selective for a specific type of eukaryotic cell. Where dendritic cells are the desired target cell, the non-native amino acid sequence can optionally recognize a protein typically found on dendritic cell surfaces. Preferred ligands which target dendritic cells recognize the CD40 cell surface protein, such as, for example, by way of a CD-40 (bi)specific antibody fragment or by way of a domain derived from the CD40L polypeptide. Where macrophages are the desired target, the non-native amino acid sequence optionally can recognize a protein typically found on macrophage cell surfaces, such as, for example, Fc receptor proteins (e.g., subtypes of Fcα, Fcγ, Fcε, etc.). Where B-cells are the desired target, the non-native amino acid sequence can recognize a protein typically found on B-cell surfaces, such as, for example, CD19 or B220.

In yet another embodiment, the adenoviral vector can comprise a chimeric virus coat protein that is not selective for a specific type of eukaryotic cell. The chimeric coat protein differs from a wild-type coat protein by an insertion of a non-native amino acid sequence into or in place of an internal coat protein sequence, or attachment of a non-native amino acid sequence to the N- or C-terminus of the coat protein. For example, a ligand comprising about five to about nine lysine residues (preferably seven lysine residues) is attached to the C-terminus of the adenoviral fiber protein via a non-functional spacer sequence. In this embodiment, the chimeric virus coat protein efficiently binds to a broader range of eukaryotic cells than a wild-type virus coat, such as described in U.S. Pat. No. 6,465,253 and International Patent Application Publication WO 97/20051.

In a preferred embodiment, the inventive method comprises an adenovirus with a coat protein and an antigen of an addictive drug conjugated to the coat protein. The antigen can be conjugated to any coat protein, such as a hexon, a fiber, or a penton base. An “antigen” of an addictive drug is a molecule that induces an immune response in a mammal against the addictive drug. An “immune response” can entail, for example, antibody production and/or the activation of immune effector cells (e.g., T cells). Thus, the antigen in the context of the invention can comprise the addictive drug or analog thereof, or a suitable portion thereof, which induces an immune response against the addictive drug. As such, the antigen can comprise any epitope of the addictive drug or analog thereof, which ideally provokes an immune response in a mammal, especially a human, against the addictive drug. By “epitope” is meant a structure that is recognized by an antibody or an antigen receptor. Epitopes also are referred to in the art as “antigenic determinants.”

Typically, the antigen is a small molecule. The term “small molecule” refers to a non-biological (i.e., non-protein, non-nucleic acid) substance or compound having a molecular weight of less than about 1,000 g/mol. Desirably, the small molecule of the invention is a hapten. By “hapten” is meant a small molecule capable of eliciting an immune response only when conjugated to a carrier substance, such as a protein, which can be processed by antigen presenting cells and presented to the immune system. Further, the hapten is characterized as the specificity-determining portion of the hapten-carrier conjugate, that is, it is capable of reacting with an antibody specific to the hapten in its free state. In a non-immunized addicted subject, there is an absence of formation of antibodies to the hapten.

The antigen can be a portion of the addictive drug, an analog or derivative of the addictive drug, or a portion thereof. By “analog” or “derivative” it is meant that the antigen has one or more atoms, functional groups, or substructures which have been replaced with different atoms, groups, or substructures. The use of an analog or derivative of an addictive drug can offer several benefits in the invention, such as, for example, to facilitate conjugation to an adenoviral coat protein or to enhance the immune response. Desirably, the analog is capable of eliciting an immune response that is equal to or greater than the immune response generated by the addictive drug from which it is derived. For example, an adenovirus comprising an analog of an addictive drug may generate antibodies having a higher titer, specificity, affinity and/or avidity for the solution conformation of the addictive drug as compared to antibodies generated in response to an adenovirus comprising the drug from which the analog is derived.

The antigen can be any addictive drug, or portion or analog thereof. Exemplary classes of addictive drugs suitable for use in the invention include, without limitation, opioids, morphine derivatives, depressants, dissociative anesthetics, cannabinoids, hallucinogens, stimulants, prescription medications, anabolic steroids, inhalants, and club drugs. Specific examples of drugs within these classes include, without limitation, nicotine, cocaine, fentanyl, heroin, morphine, opium, oxycodone, hydrocodone, ketamine, PCP, barbiturates, benzodiazepines, flunitrazepam, GHB, methaqualone, hashish, marijuana, LSD, mescaline, psilocybin, amphetamine, cocaine, MDMA, methamphetamine, and methylphenidate.

A preferred antigen of the invention is nicotine. Several nicotine haptens, carriers, and methods of conjugation have been described. Nicotine can be conjugated to an adenovirus using any suitable method known in the art. For example, nicotine can be conjugated to an adenoviral coat protein via a linker at the 6-position or at the 1-position as previously described for nicotine-BSA conjugates and nicotine-KLH conjugates (see, e.g., Matsushita et al., Biochem. Biophys. Res. Comm., 57: 1006-1010 (1974), Castro et al., Eur. J. Biochem., 104: 331-340 (1980), Noguchi et al., Biochem. Biophys. Res. Comm., 83: 83-86 (1978), and Isomura et al., J. Org. Chem., 66: 4115-4121 (2001)). Nicotine also can be conjugated to an adenovirus via the pyridine ring as described in International Patent Application Publication WO 99/61054, or the pyrrolidine ring as described in U.S. Pat. No. 6,232,082.

The antigen also can be an analog of nicotine. Suitable nicotine analogs include any nicotine analog that induces an immune response in a mammal (humoral or cell-mediated). Nicotine analogs are known in the art (see, e.g., Cerny et al., Onkologie, 25: 406-411 (2002); Lindblom et al., Respiration, 69: 254-260 (2002); de Villiers et al., Respiration, 69: 247-253 (2002); Tuncok et al., Exp. Clin. Psychopharmacol., 9: 228-234 (2001); Hieda et al., Int. J. Immunopharmacol., 22: 809-819 (2000); Pentel et al., Pharmacol. Biochem. Behay., 65: 191-198 (2000); Isomura et al., J. Org. Chem., 66: 4115-4121 (2001); and Meijler et al., J. Am. Chem. Soc., 125: 7164-7165 (2003). For example, the nicotine analog can be N-succinyl-6-amino-(+/−)-nicotine (Castro et al., Biochem. Biophys. Res. Commun., 67: 583-589 (1975)), 6-(sigma-aminocapramido)-(+/−)-nicotine (Noguchi et al., Biochem. Biophys. Res. Comm., 83: 83-86 (1978)), O-succinyl-3′-hydroxymethyl-nicotine (Langone et al., Biochemistry, 12: 5025-5030 (1973); and Meth. Enzymol., 84: 628-640 (1982)), or 3′-(hydroxymethyl)-nicotine hemisuccinate (Langone et al., supra, Abad et al., Anal. Chem., 65: 3227-3231 (1993)). Additional examples of nicotine analogs suitable for use in the invention are described in U.S. Pat. Nos. 6,232,082 and 6,932,971. In a preferred embodiment, the nicotine analog is AM3. Novel nicotine analogs also can be used in the context of the invention, and examples of novel nicotine analogs are described herein (see Examples).

In another embodiment, the antigen can be cocaine. For example, the free acid of cocaine, diazonium salts of benzoyl cocaine and benzoyl ecognine, or the para-imino ester derivatives of cocaine and norcocaine (described in, e.g., U.S. Pat. Nos. 4,123,431; 4,197,237; and 6,932,971) can be conjugated to an adenovirus. Additional examples of cocaine analogs suitable for use as an antigen of the invention are described in U.S. Pat. No. 5,876,727. In addition, the antigen can be an acylated ecgonine methyl ester, a succinylated ecgonine methyl ester, a succinylated norcocaine, or benzoyl ecgonine. Preferably, the antigen is the cocaine analog GNC or the cocaine analog GNE.

Methods of conjugating a hapten to a protein carrier are well known in the art, and can be readily adapted to the conjugation of an addictive drug antigen to an adenoviral coat protein. Such methods are described in, e.g., Sambrook et al., supra, Ausubel, et al., supra, and Harlow and Lane, “Antibodies: A Laboratory Manual,” Cold Spring Harbor Laboratory, Cold Spring Harbor, N.Y. (1988).

There are a large number of functional groups which can be used to facilitate the conjugation of a hapten to an adenoviral coat protein. These include functional moieties such as carboxylic acids, anhydrides, mixed anhydrides, acyl halides, acyl azides, alkyl halides, N-maleimides, imino esters, isocyanates, amines, thiols, isothiocyanates, and others known in the art. These moieties are capable of forming a covalent bond with a reactive group of a an adenoviral coat protein. Depending upon the functional moiety used, the reactive group may be the free amino group of a lysine residue or a free thiol group of a cysteine residue on an adenoviral coat protein which, when reacted, results in amide, amine, thioether, amidine urea, or thiourea bond formation. One of ordinary skill in the art will recognize that other suitable activating groups and conjugation techniques can be used, such as those described in Wong, Chemistry of Protein Conjugation and Cross-Linking (CRC Press, Inc., 1991); Hermanson, Bioconjugate Techniques (Academic Press, 1996); and Dick and Beurret, “Conjugate Vaccines,” Contrib. Microbiol. Immunol., 10: 48-114 (Karger, Basal, 1989).

The antigen can be conjugated to an adenoviral coat protein using a homo-bifunctional cross-linker, such as glutaraldehyde, DSG, BM[PEO]₄, or BS³, which has functional groups reactive towards amine groups or carboxyl groups of an adenoviral coat protein. Desirably, the antigen is conjugated to an adenoviral coat protein by way of chemical cross-linking using a hetero-bifunctional cross-linker. Generally, in the first step of the procedure (often referred to as derivatization) the adenovirus is reacted with the cross-linker, thereby resulting in an adenovirus containing one or more activated coat proteins. In the second step, unreacted cross-linker is removed using methods such as gel filtration or dialysis. In the third step, the antigen is reacted or “coupled” with the activated coat protein. In an optional fourth step, unreacted antigen is removed.

Several hetero-bifunctional cross-linkers are known in the art. For example, the hetero-bifunctional cross-linker can contain a functional group which reacts with the free amino group of lysine residues of an adenoviral coat protein, and a functional group which reacts with a cysteine residue or sulfhydryl group present on the antigen, thereby leading to the formation of a thioether linkage. The cysteine residue or sulfhydryl group can be naturally present on the antigen, made available for reaction by reduction, or engineered or attached on the antigen (e.g., a hapten) and optionally made available for reaction by reduction. Several such hetero-bifunctional cross-linkers are known in the art, and include, for example, SMPH, Sulfo-MBS, Sulfo-EMCS, Sulfo-GMBS, Sulfo-SIAB, Sulfo-SMPB, Sulfo-SMCC, SVSB, and SIA, which are commercially available from, for example, Pierce Thermo Fisher Scientific (Rockford, Ill., USA).

A preferred linker is a succinyl functional moiety, which forms succinimidyl ester cross-links of the antigen to epsilon amino groups exposed on an adenoviral capsid surface (Leopold et al., Hum. Gene Ther., 9: 367-378 (1998) and Miyazawa et al., J. Virol., 73: 6056-6065 (1999)). Examples of linkers comprising a succinyl functional moiety are N-hydroxysulfosuccinimide (Sulfo-NHS) and its uncharged analog N-hydroxysuccinimide (NHS), which are used to convert carboxyl groups to amine-reactive Sulfo-NHS esters. The presence of Sulfo-NHS esters increases the efficiency of coupling reactions mediated by carbodiimide compounds, such as EDAC (1-ethyl-3-[3-dimethylaminopropyl]carbodiimide hydrochlroide), which couple carboxyl groups to primary amines. Maleimides, which conjugate to sulfhydryl groups, can also be used to conjugate an antigen of an addictive drug to a coat protein of an adenovirus.

The amount of antigen that is conjugated per adenoviral particle is one factor which regulates the immune response induced by the antigen. Various strategies which are known in the art can be used in accordance with the invention to optimize the amount of conjugated antigen. For example, the extent of derivatization of the adenoviral coat protein with cross-linker can be influenced by varying experimental conditions such as the concentration of each of the reaction partners, the excess of one reagent over the other, the pH, the temperature, and the ionic strength. Similarly, the degree of coupling, i.e., the amount of antigen per adenoviral particle, can be adjusted by varying the experimental conditions described above to match the requirements of the vaccine. The degree of coupling can also be expressed as the amount of antigen per adenoviral capsomere. By “capsomere” is meant a morphological subunit of the adenovirus capsid formed from the major coat proteins. The outer capsid of an adenoviral virion consists of 252 capsomeres (see, e.g., van Oostrum and Burnett, J. Virol., 56: 439-448 (1985)). The ratio of adenoviral capsomere to antigen molecule (i.e., Ad:Ag) utilized to prepare the inventive adenovirus-antigen conjugates can be, 1:1 or more, e.g., 1:3 or more, 1:10 or more, or 1:30 or more. Alternatively, or in addition, the Ad:Ag ratio can be 1:1000 or less, e.g., 1:500 or less, 1:300 or less, or 1:100 or less. Thus, the Ad:Ag ratio can be bounded by any two of the above endpoints. For example, the Ad:Ag ratio can be 1:1-1:1000, 1:3-1:500, 1:10-1:300, 1:10-1:100, or 1:30-1:100.

Once the adenoviral particles have been conjugated to an addictive drug antigen, the relative extent of conjugation can be determined by measuring the absorbance of free antigen at its absorbance maximum (λₘₐₓ) and applying Beer's Law to determine the molar concentration of the antigen. The calculation requires that the absorbance of adenovirus prior to conjugation and after conjugation be measured in order to determine the deflection from baseline absorbance specifically attributable to the conjugated antigen. The relative extent of conjugation also can be monitored by MALDI-TOF MS. Achieving a conjugation rate of 0.3 to 2.0 antigen molecules per capsomere (or approximately 80 to 500 antigen molecules per adenoviral particle) would be comparable to the conjugation levels observed for the fluorophore, Cy3, as previously described (Leopold et al., Hum. Gene Ther., 9: 367-378 (1998)). An “overconjugated” adenovirus can be beneficial for hapten-mediated vaccination. Therefore, the number of antigen molecules per adenoviral particle in an overconjugated adenovirus can be 40 or more, e.g., 80 or more, 120 or more, or 200 or more. Alternatively, or in addition, the number of antigen molecules per adenoviral particle in an overconjugated adenovirus can be 1000 or less, e.g., 750 or less, 500 or less, or 300 or less. Thus, the number of antigen molecules per adenoviral particle can be bounded by any two of the above endpoints. For example, the number of antigen molecules per adenoviral particle in an overconjugated adenovirus can be 40-1000, 80-750, 120-500, 200-500, or 200-300.

Assuming equal affinity for antigen, there may be a direct correlation between antibody titer and vaccine efficacy. Therefore, increasing the amount of antigen that is conjugated to the adenovirus may enhance the immunogenicity thereof. Exposed lysine residues on an adenoviral capsid protein (e.g., hexon) provide a free amine group that is a target for conjugation to carboxylate group-containing antigens, and many of the aforementioned cross-linking reagents react preferentially with lysine residues.

When an antigen is conjugated to an adenoviral coat protein via lysine residues, it may be advantageous to add or to remove one or more lysine residues to the adenoviral coat protein in order to regulate antigen conjugation. The invention further provides a method of inducing an immune response against an addictive drug in a human comprising administering to a human an adenovirus-antigen conjugate comprising an adenovirus with a coat protein and an antigen of an addictive drug conjugated to the coat protein of the adenovirus, wherein the coat protein comprises at least one non-native lysine residue, and whereby the antigen is presented to the immune system of the human to induce an immune response against the addictive drug in the human. The number of non-native lysine residues can be 1 or more, e.g., 3 or more, 5 or more, or 7 or more. Alternatively, or in addition, the number of non-native lysine residues can be 25 or less, e.g., 20 or less, 15 or less, or 10 or less. Thus, the number of non-native lysine residues can be bounded by any two of the above endpoints. For example, the number of non-native lysine residues can be 1-25, 3-20, 5-10, 5-15, or 7-10.

The invention also provides a method of inducing an immune response against an addictive drug in a human comprising administering to a human an adenovirus-antigen conjugate comprising an adenovirus with a coat protein and an antigen of an addictive drug conjugated to the coat protein of the adenovirus, wherein at least one native lysine residue is absent from the coat protein, and whereby the antigen is presented to the immune system of the human to induce an immune response against the addictive drug in the human. The removal of at least one native lysine residue may effected be by deletion or substitution, and can be in the context of an otherwise unmodified coat protein. Alternatively, the removal of at least one native lysine residue can be in the context of a coat protein that also comprises at least one non-native lysine residue as described above. The number of absent native lysine residues can be in the same ranges described above for non-native lysine residues present in the coat protein.

The coat protein that comprises at least one non-native lysine residue or lacks at least one native lysine residue can be any adenovirus coat protein (e.g., fiber, penton base, or hexon). Preferably, the coat protein that comprises at least one non-native lysine or in which at least one native lysine residue is absent is a hexon protein. When non-native lysine residues are added to a hexon protein, it is preferred that the lysine residues are incorporated into one or more flexible loops of the hexon protein. Standard molecular biology techniques which are well known in the art can be utilized to generate modified coat proteins in accordance with the invention (see, e.g., Sambrook et al., supra, and Ausubel, et al., supra).

In another embodiment of the invention, the adenovirus further comprises one or more transgenes, each encoding a protein that stimulates one or more cells of the immune system. By “transgene” is meant any heterologous nucleic acid sequence that can be carried by an adenoviral vector and expressed in a cell. A “heterologous nucleic acid sequence” is any nucleic acid sequence that is not obtained from, derived from, or based upon a naturally occurring nucleic acid sequence of the adenovirus. The adenovirus can comprise at least one transgene as described herein, i.e., the adenovirus can comprise one transgene as described herein or more than one transgene as described herein (i.e., two or more of transgenes). The transgene preferably encodes a protein (i.e., one or more nucleic acid sequences encoding one or more proteins). An ordinarily skilled artisan will appreciate that any type of nucleic acid sequence (e.g., DNA, RNA, and cDNA) that can be inserted into an adenovirus can be used in connection with the invention.

In a preferred embodiment, the transgene encodes a protein that enhances the immune response in an animal. For example, the transgene can encode a protein that elevates the humoral immune response to haptens on the adenovirus capsid. Alternatively, the transgene can encode a protein that enhances the cell-mediated immune response to the adenovirus-antigen conjugate. The one or more transgenes can encode, for example, a dendritic cell activating protein (e.g., CD40L), a B cell activating protein (e.g., B-cell Activating Factor (BAFF)), a T cell activating protein (e.g., IL-15), or combinations thereof. Preferably, the one or more transgenes encode a protein that stimulates B cell activity. Most preferably, the adenovirus comprises a transgene encoding BAFF.

The one or more transgenes in the adenovirus desirably are present as part of an expression cassette, i.e., a particular nucleotide sequence that possesses functions which facilitate subcloning and recovery of a nucleic acid sequence (e.g., one or more restriction sites) or expression of a nucleic acid sequence (e.g., polyadenylation or splice sites). The one or more transgenes can be located in any suitable region of the adenovirus. Preferably, the one or more transgenes are located in the E1 region (e.g., replaces the E1 region in whole or in part). For example, the E1 region can be replaced by one or more expression cassettes comprising a transgene. Additionally or alternatively, the one or more transgenes can be located in the E4 region (e.g., replaces the E4 region in whole or in part).

Preferably, the transgene is operably linked to (i.e., under the transcriptional control of) one or more promoter elements. Techniques for operably linking sequences together are well known in the art. A “promoter” is a DNA sequence that directs the binding of RNA polymerase and thereby promotes RNA synthesis. A nucleic acid sequence is “operably linked” to a promoter when the promoter is capable of directing transcription of the nucleic acid sequence. A promoter can be native or non-native to the nucleic acid sequence to which it is operably linked.

Any promoter (i.e., whether isolated from nature or produced by recombinant DNA or synthetic techniques) can be used in connection with the invention to provide for transcription of a heterologous nucleic acid sequence (e.g., a transgene). The promoter preferably is capable of directing transcription in a eukaryotic (desirably mammalian) cell. Any suitable promoter sequence can be used in the context of the invention. In this respect, the transgene can be operably linked to a viral promoter. Suitable viral promoters include, for instance, cytomegalovirus (CMV) promoters (described in, for example, U.S. Pat. Nos. 5,168,062 and 5,385,839, and GenBank accession number X17403), promoters derived from human immunodeficiency virus (HIV), such as the HIV long terminal repeat promoter, Rous sarcoma virus (RSV) promoters, such as the RSV long terminal repeat, mouse mammary tumor virus (MMTV) promoters, HSV promoters, such as the herpes thymidine kinase promoter (Wagner et al., Proc. Natl. Acad. Sci., 78: 144-145 (1981)), promoters derived from SV40 or Epstein Barr virus, and the like.

Alternatively, the transgene can be operably linked to a cellular promoter, i.e., a promoter that drives expression of a cellular protein. Preferred cellular promoters for use in the invention will depend on the desired expression profile of the transgene. In one aspect, the cellular promoter is preferably a constitutive promoter that works in a variety of cell types, such as cells of the immune system (e.g., dendritic cells). Suitable constitutive promoters can drive expression of genes encoding transcription factors, housekeeping genes, or structural genes common to eukaryotic cells. Constitutively active cellular promoters are known in the art and include, for example, the Ying Yang 1 (YY1) promoter, the JEM-1 promoter, the ubiquitin promoter, and the elongation factor alpha (EF1α) promoter.

Instead of being a constitutive promoter, the promoter can be an inducible promoter, i.e., a promoter that is up- and/or down-regulated in response to an appropriate signal. A promoter can be up-regulated by a radiant energy source or by a substance that distresses cells. For example, a promoter can be up-regulated by drugs, hormones, ultrasound, light activated compounds, radiofrequency, chemotherapy, and cryofreezing. Thus, the promoter sequence that regulates expression of the transgene sequence can contain at least one heterologous regulatory sequence responsive to regulation by an exogenous agent. Suitable inducible promoter systems include, but are not limited to, the IL-8 promoter, the metallothionine inducible promoter system, the bacterial lacZYA expression system, the tetracycline expression system, and the T7 polymerase system. Further, promoters that are selectively activated at different developmental stages (e.g., globin genes are differentially transcribed from globin-associated promoters in embryos and adults) can be employed. In another embodiment, the promoter can be a tissue-specific promoter, i.e., a promoter that is preferentially activated in a given tissue and results in expression of a gene product in the tissue where activated. A tissue-specific promoter suitable for use in the invention can be chosen by the ordinarily skilled artisan based upon the target tissue or cell-type.

Operable linkage of a transgene to a promoter is within the skill of the art, and can be accomplished using routine recombinant DNA techniques, such as those described in, for example, Sambrook et al., supra, and Ausubel et al., supra.

To optimize protein production, preferably the transgene further comprises a polyadenylation site 3′ of the coding sequence of the transgene. Any suitable polyadenylation sequence can be used, including a synthetic optimized sequence, as well as the polyadenylation sequence of BGH (Bovine Growth Hormone), mouse globin D (MGD), polyoma virus, TK (Thymidine Kinase), EBV (Epstein Barr Virus), and the papillomaviruses, including human papillomaviruses and BPV (Bovine Papilloma Virus). A preferred polyadenylation sequence is the SV40 (Human Sarcoma Virus-40) polyadenylation sequence. Also, preferably all the proper transcription signals (and translation signals, where appropriate) are correctly arranged such that the each nucleic acid sequence is properly expressed in the cells into which it is introduced. If desired, the heterologous nucleic acid sequence also can incorporate splice sites (i.e., splice acceptor and splice donor sites) to facilitate mRNA production.

An antibody produced in a mammal in response to the administration of the adenovirus-antigen conjugate can be isolated and used for a variety of purposes. When the antibody is isolated from a non-human mammal, the antibody can be humanized for subsequent administration to a human. “Humanized” forms of non-human (e.g., murine) antibodies are chimeric antibodies which contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or non-human primate having the desired specificity, affinity, and capacity. In some instances, framework region residues of the human immunoglobulin are replaced by corresponding non-human residues. Furthermore, humanized antibodies can comprise residues which are not found in the recipient antibody or in the donor antibody. These modifications are made to further refine antibody performance. A humanized antibody can comprise substantially all of at least one and, in some cases two, variable domains, in which all or substantially all of the hypervariable regions correspond to those of a non-human immunoglobulin and all, or substantially all, of the framework regions are those of a human immunoglobulin sequence. The humanized antibody optionally also will comprise at least a portion of an immunoglobulin constant region, typically that of a human immunoglobulin. For further details, see Jones et al., Nature, 321: 522-525 (1986), Reichmann et al., Nature, 332: 323-329 (1988), and Presta, Curr. Op. Struct. Biol., 2: 593-596 (1992). Methods of preparing humanized antibodies are generally well known in the art and can readily be applied to the antibodies produced by the methods described herein.

The invention also provides a method of reducing the effect of an addictive drug in a human, which method comprises administering to the human an adenoviral vector comprising a nucleic acid sequence which encodes an antibody directed against the addictive drug and which is operably linked to a promoter, wherein the nucleic acid sequence is expressed in the human to reduce the effect of the addictive drug. By “reduce the effect” is meant, for example, a reduction in the physiological effects of the addictive drug, a reduction in the reward or pleasure associated with use of the addictive drug, or a reduction in the likelihood of regaining an addiction to the drug. Descriptions of the adenoviral vector, addictive drugs, and promoters set forth above in connection with other embodiments of the invention are also applicable to those same aspects of the aforesaid method.

In the context of the invention, the nucleic acid sequence which encodes an antibody directed against an addictive drug can encode any such antibody (or portion thereof) known in the art. For example, the nucleic acid sequence can encode the cocaine-binding monoclonal antibody GNC92H2 (Redwan et al., Biotechnol. Bioeng., 82(5): 612-8 (2003)) or the nicotine-binding monoclonal antibody Nic12 (Beerli et al., Proc. Arad Acad. Sci. USA, 105(38): 14336-14341 (2008)). In another embodiment, a nucleic acid sequence encoding an antibody which has been isolated from a mammal vaccinated with the adenovirus-antigen conjugate of the invention can be used. Independent of the source of the antibody against the addictive drug, the nucleic acid sequence encoding an antibody can encode a whole antibody molecule, or any antigen-binding fragment thereof, such as Fab, Fab′, F(ab′)2, single-chain Fvs (scFv), single-chain antibodies, disulfide-linked Fvs, or fragments comprising either a V_{L} or V_{H} domain. Moreover, the nucleic acid sequence desirably encodes a polyclonal or monoclonal antibody, but preferably, the nucleic acid sequence encodes a monoclonal antibody.

In addition, the invention provides an adenovirus-antigen conjugate comprising an adenovirus with a coat protein and an antigen of an addictive drug conjugated to the coat protein of the adenovirus, as well as an adenoviral vector comprising a nucleic acid sequence which encodes an antibody directed against an addictive drug, which nucleic acid sequence is operably linked to a promoter, wherein the nucleic acid sequence can be expressed in a human to produce the antibody. Descriptions of the adenovirus, adenoviral vector, addictive drug antigen, conjugation, transgene, etc. set forth above in connection with embodiments of the inventive methods also are applicable to those same aspects of the aforesaid adenovirus-antigen conjugate and adenoviral vector.

The invention also provides compositions comprising (a) the adenovirus-antigen conjugate or the adenoviral vector and (b) a carrier therefor. Preferably, the composition is a pharmaceutically acceptable (e.g., physiologically acceptable) composition, which comprises a carrier, preferably a pharmaceutically (e.g., physiologically acceptable) carrier and the adenoviral vector. Any suitable carrier can be used within the context of the invention, and such carriers are well known in the art. The choice of carrier will be determined, in part, by the particular site to which the composition is to be administered and the particular method used to administer the composition. The composition preferably is free of replication-competent adenovirus. The composition optionally can be sterile with the exception of the adenovirus-antigen conjugate or adenoviral vector described herein.

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. Preferably, the carrier is a buffered saline solution. More preferably, the adenovirus-antigen conjugate or adenoviral vector is administered in a composition formulated to protect the adenovirus-antigen conjugate or adenoviral vector from damage prior to administration. For example, the composition can be formulated to reduce loss of the adenovirus-antigen conjugate or adenoviral vector on devices used to prepare, store, or administer the adenovirus-antigen conjugate or adenoviral vector, such as glassware, syringes, or needles. The composition can be formulated to decrease the light sensitivity and/or temperature sensitivity of the adenovirus-antigen conjugate or adenoviral vector. To this end, the composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the adenovirus-antigen conjugate or adenoviral vector, facilitate administration, and increase the efficiency of the inventive method. Formulations for adenovirus-containing or adenoviral vector-containing compositions are further described in, for example, U.S. Pat. No. 6,225,289, U.S. Pat. No. 6,514,943, U.S. Patent Application Publication 2003/0153065 A1, and International Patent Application Publication WO 00/34444.

A composition also can be formulated to enhance transduction efficiency. In addition, one of ordinary skill in the art will appreciate that the adenovirus-antigen conjugate or adenoviral vector can be present in a composition with other therapeutic or biologically-active agents. For example, factors that control inflammation, such as ibuprofen or steroids, can be part of the composition to reduce swelling and inflammation associated with in vivo administration of the adenovirus-antigen conjugate or adenoviral vector. Immune system stimulators or adjuvants, e.g., interleukins, lipopolysaccharide, and double-stranded RNA, can be administered to enhance or modify any immune response to the antigen. Antibiotics, i.e., microbicides and fungicides, can be present to treat existing infection and/or reduce the risk of future infection, such as infection associated with gene transfer procedures.

The adenovirus-antigen conjugate preferably is administered to a mammal (e.g., a human), whereupon the antigen induces an immune response against the addictive drug. The immune response can be a humoral immune response, a cell-mediated immune response, or, desirably, a combination of humoral and cell-mediated immunity. Similarly, the adenoviral vector preferably is administered to a mammal (e.g., a human), whereupon the nucleic acid sequence encoding an antibody to the addictive drug is expressed so as to produce an antibody to the addictive drug. Ideally, the immune response or produced antibody provides a clinical benefit upon exposure to the addictive drug. By “clinical benefit” is meant, for example, a reduction in the physiological effects of the addictive drug, a reduction in the reward or pleasure associated with use of the addictive drug, or a reduction in the likelihood of regaining an addiction to the drug. However, a clinical benefit is not required in the context of the invention. The inventive method further can be used for antibody production and harvesting.

Administering the adenovirus-antigen conjugate or adenoviral vector can be one component of a multistep regimen for inducing an immune response in a mammal. In particular, the inventive method can represent one arm of a prime and boost immunization regimen. The inventive method, therefore, can comprise administering to the mammal a priming adenovirus-antigen conjugate or adenoviral vector prior to administering, or “boosting,” with the priming or a different adenovirus-antigen conjugate or adenoviral vector. More than one boosting composition comprising an adenovirus-antigen conjugate or adenoviral vector can be provided in any suitable timeframe (e.g., at least about 1 week, 2 weeks, 4 weeks, 8 weeks, 12 weeks, 16 weeks, or more following priming) to maintain immunity.

Any route of administration can be used to deliver the adenovirus-antigen conjugate or adenoviral vector to the mammal. Indeed, although more than one route can be used to administer the adenovirus-antigen conjugate or adenoviral vector, a particular route can provide a more immediate and more effective reaction than another route. Preferably, the adenovirus-antigen conjugate or adenoviral vector is administered via intramuscular injection. A dose of adenovirus-antigen conjugate or adenoviral vector also can be applied or instilled into body cavities, absorbed through the skin (e.g., via a transdermal patch), inhaled, ingested, topically applied to tissue, or administered parenterally via, for instance, intravenous, peritoneal, or intraarterial administration.

The adenovirus-antigen conjugate or adenoviral vector can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the adenoviral vector. The adenovirus-antigen conjugate or adenoviral vector also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of adenovirus-antigen conjugate or adenoviral vector administered to the mammal will depend on a number of factors, including the size of a target tissue, the extent of any side-effects, the particular route of administration, and the like. The dose ideally comprises an “effective amount” of adenovirus-antigen conjugate or adenoviral vector, i.e., a dose of adenovirus-antigen conjugate or adenoviral vector which provokes a desired immune response in the mammal or production of the desired quantity of antibodies in the mammal. The desired immune response can entail production of antibodies, protection upon subsequent challenge, immune tolerance, immune cell activation, and the like. Similarly, the desired quantity of antibodies can provide protection upon subsequent challenge, immune tolerance, and the like. Desirably, a single dose of adenovirus-antigen conjugate or adenoviral vector comprises at least about 1×10⁵ particles (which also is referred to as particle units) of the adenovirus-antigen conjugate or adenoviral vector. The dose preferably is at least about 1×10⁶ particles (e.g., about 1×10⁶-1×10¹² particles), more preferably at least about 1×10⁷ particles, more preferably at least about 1×10⁸ particles (e.g., about 1×10⁸-1×10¹¹ particles), and most preferably at least about 1×10⁹ particles (e.g., about 1×10⁹-1×10¹⁰ particles) of the adenovirus-antigen conjugate or adenoviral vector. The dose desirably comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ particles) of the adenovirus-antigen conjugate or adenoviral vector. In other words, a single dose of adenovirus-antigen conjugate or adenoviral vector can comprise, for example, about 1×10⁶ particle units (pu), 2×10⁶ pu, 4×10⁶ pu, 1×10⁷ pu, 2×10⁷ pu, 4×10⁷ pu, 1×10⁸ pu, 2×10⁸ pu, 4×10⁸ pu, 1×10⁹ pu, 2×10⁹ pu, 4×10⁹ pu, 1×10¹⁰ pu, 2×10¹⁰ pu, 4×10¹⁰ pu, 1×10¹¹ pu, 2×10¹¹ pu, 4×10¹¹ pu, 1×10¹² pu, 2×10¹² pu, or 4×10¹² pu of the adenovirus-antigen conjugate or adenoviral vector.

The adenovirus-antigen conjugate or adenoviral vector can be administered in conjunction with counseling and/or one or more additional agents that prevent or treat drug addiction. The additional agent may treat withdrawal symptoms, facilitate quitting, or prevent relapse. When the adenovirus is conjugated to a nicotine hapten, the additional agent can be, for example, an anti-depressant, a nicotine receptor modulator, a cannabinoid receptor antagonist, an opioid receptor antagonist, a monoamine oxidase inhibitor, an anxiolytic, or any combination of these agents. Preferably, the additional agent is an anti-depressant selected from the group consisting of bupropion, doxepin, desipramine, clomipramine, imipramine, nortriptyline, amitriptyline, protriptyline, trimipramine, fluoxetine, fluvoxamine, paroxetine, sertraline, phenelzine, tranylcypromine, amoxapine, maprotiline, trazodone, venlafaxine, mirtazapine, and pharmaceutically active salts or optical isomers thereof. When the adenovirus is conjugated to a cocaine hapten, the additional agent can be, for example, an opioid receptor antagonist, an anti-depressant such as desipramine or fluoxetine, or an agent which regulates the dopaminergic system (e.g., bromocriptine or buprenorphine).

The following examples further illustrate the invention but should not be construed as in any way limiting its scope.

### Example 1

This example demonstrates a method of inducing an immune response in a mammal using an adenoviral vector-fluorophore conjugate.

The fluorophore, Cy3, was conjugated to the capsid of an adenovirus serotype 5 vector (Cy3Ad). Cy3Ad was delivered to mice by intravenous administration, and mouse serum was collected after three weeks.

Western blotting was performed to determine whether anti-Cy3 antibodies were present in the immunized mouse serum samples. Briefly, annexin V or annexin V conjugated to Cy3 (Cy3-Annexin V) was loaded onto SDS-PAGE gels. After electrophoresis, the proteins were visualized by staining with Coomassie blue, or were transferred to nitrocellulose for Western blotting using control mouse serum or Cy3Ad vaccinated serum as the primary antibody. The results of the Western blots demonstrated that Cy3Ad-vaccinated mouse serum, but not the control PBS-injected mouse serum, contained antibodies specific for Cy3.

The results of this example demonstrate that an adenovirus conjugated to a small molecule hapten can be utilized to induce an immune response in a mammal.

### Example 2

This example demonstrates a method of inducing humoral immunity in a mammal using an adenovirus comprising a nicotine antigen conjugated to the capsid.

Mice were immunized with nicotine-conjugated Ad or, as a negative control, an unconjugated mixture of Ad and nicotine. At 2 weeks post-immunization, sera was collected and analyzed for nicotine-specific antibodies by Western analysis with immune mouse serum as the primary antibody against nicotine-conjugated target antigens or control antigens. Mice immunized with the unconjugated mixture of Ad and nicotine did not develop anti-nicotine humoral immunity as determined by Western blotting. Mice immunized with nicotine-conjugated Ad did not develop antibodies reactive with the negative control antigens BSA or KLH. In contrast, serum from mice immunized with nicotine-conjugated Ad was strongly reactive against nicotine-conjugated Ad, nicotine-conjugated BSA and nicotine-conjugated KLH as determined by Western blotting. A low level of reactivity against adenovirus was also detected.

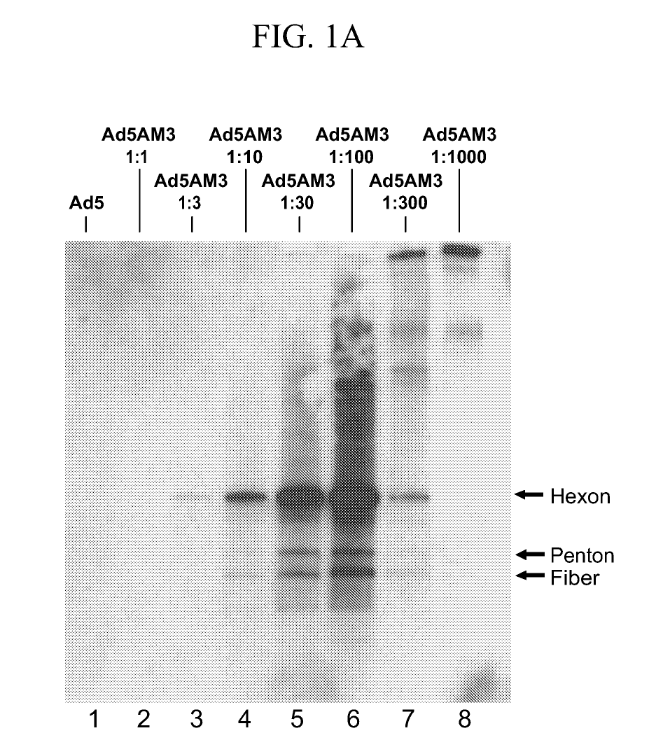

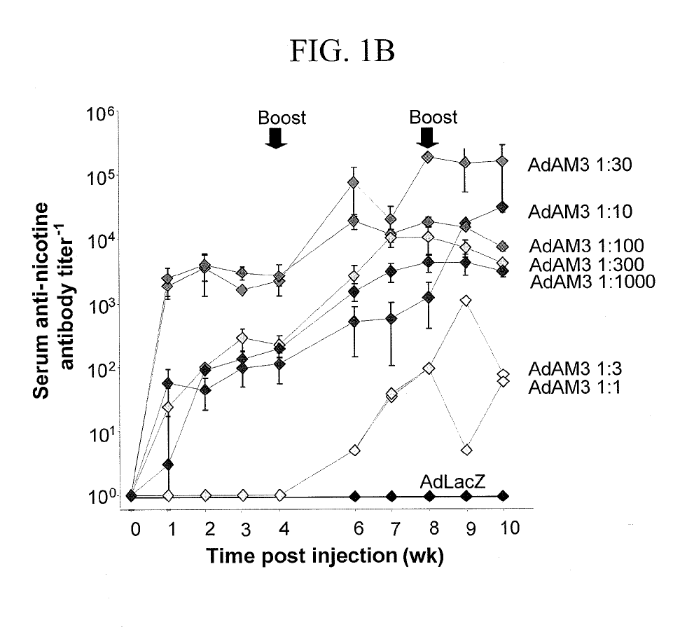

A nicotine analog, AM3, was conjugated to the surface of a replication-defective human serotype 5 gene transfer vector (E1/E3-deficient) using Sulfo-NHS and EDC chemistry for use as a nicotine vaccine (Ad5AM3). The precise ratio of Ad5 to AM3 required for eliciting an optimal immune response is unknown. Therefore, a panel of Ad5AM3 conjugates was generated at various ratios of Ad5 capsomere to AM3 molecule (i.e., Ad5:AM3), and the presence of AM3 on the major virion proteins was examined by Western analysis with a nicotine-specific antibody. The results of this experiment demonstrated that Ad5:AM3 conjugates prepared at a ratio of 1:3, 1:10, 1:30, 1:100, and 1:300 were reactive with a nicotine-specific antibody (FIG. 1A). Most of the immunoreactivity was associated with the hexon protein of the adenovirus, although some immunoreactivity also was associated with the penton and fiber proteins of the adenovirus (FIG. 1A).

Mice were immunized with the panel of Ad5AM3 conjugates to elicit AM3-specific immunity. Compared to mice immunized with an unconjugated control vector, immunization with Ad5AM3 conjugates at Ad5:AM3 ratios >1:3 elicited high levels of AM3-specific antibodies (FIG. 1B).

The results of this example demonstrate that conjugation of nicotine or a nicotine analog to an adenovirus can be used to induce anti-nicotine humoral immunity in a mammal.

### Example 3

The example describes the generation of adenovirus-nicotine conjugates.

In one series of experiments, two constrained nicotine analogs (i.e., compounds 1 and 2 depicted below of Meijler et al., J. Am. Chem. Soc., 125: 7164-7165 (2003)) will be synthesized, and a linker (“R”, below) will be added to create “CNA” and “CNI,” as illustrated below:

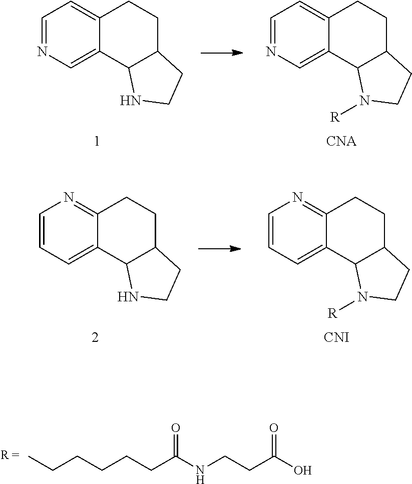

In addition, a linker will be added to a non-constrained nicotine analog, i.e., trans-S′-hydroxymethylnicotine (#H948175, Toronto Research Chemicals, North York, ON, Canada) via the hydroxyl group, allowing cross-linking to proteins. All three haptens will be conjugated to BSA for analytical studies, to KLH or ovalbumin for attachment to 40 nm polystyrene beads, and to adenovirus capsids for vaccination studies. Individual products of organic synthesis will be confirmed by acquiring ¹H NMR spectra on Bruker AMX-600 (600 MHz), AMX-500 (500 MHz), or AMX-400 (400 MHz) spectrometer, and ¹³C NMR spectra on a Bruker AMX-500 (125.7 MHz) or AMX-400 (100.6 MHz) spectrometer. The extent of nicotine conjugation will be quantified using MALDI-TOF on a VG ZAB-VSE instrument. After conjugation, sites of nicotine conjugation to adenovirus will be assessed by SDS-PAGE and Western blotting using anti-nicotine antisera.

To determine the viability of the nicotine-adenovirus conjugate, two assays will be performed: gene expression and intracellular trafficking, as previously described (Vincent et al., J. Virol., 75: 1516-1521 (2001)). The assays are complementary in that the assays test successful gene expression and successful cell association independently. To test gene expression, A549 cells will be infected with equal doses of nicotine-conjugated and unconjugated adenovirus vector carrying the β-galactosidase transgene. For β-galactosidase expression studies, A549 cells (10⁵ cells) in 12-well tissue culture plates (4 wells per condition) will be infected with 5×10⁸ particles in 500 μL of binding buffer (50% Modified Eagle Medium, 2×, 1% BSA, 10 mM HEPES, pH 7.3). After one hour, cells will be washed three times with sterile PBS and returned to culture medium for 24 hours before harvesting and analysis. β-galactosidase transgene expression will be evaluated in cell lysates 24 hours following infection using quantitative chemiluminescent detection of enzyme activity (Tropix, Inc., Bedford, Mass.). Data will be expressed as activity per mg protein determined using the BCA reagent (Bio-Rad, Hercules, Calif.).

To test trafficking, A549 cells will be infected with equal doses of nicotine-conjugated and unconjugated adenovirus vector and assessed using indirect immunofluorescence using anti-nicotine and anti-adenovirus antibodies. A549 cells (10⁵ cells) plated in coverslip-bottom dishes (Leopold et al., Hum. Gene Ther., 9: 367-378 (1998)) will be infected with a small volume (30 mL) of highly concentrated virus (10″ particles/mL) in binding buffer for a very short period (ten minutes) followed by washing and incubation. This pulse-labeling protocol results in high occupancy of viral receptors at the cell surface that traffic through the cell as a wave, reaching the nucleus in approximately one hour (see e.g., Leopold et al., In: Vo-Dinh T, ed. Nanotechnology in Biology and Medicine, Taylor and Francis, Inc/CRC Press Inc., London, UK (2006)). Hapten and adenovirus capsids will be located independently using indirect immunofluorescence. After one hour, cells will be fixed (4% paraformaldehyde), blocked, and stained with primary antibodies against anti-nicotine antibodies or murine anti-adenovirus immune sera prepared against either serotype 5 or serotype C7. Primary antibodies will be detected using appropriate fluorescently-labeled secondary antibodies (Jackson Immunoresearch, West Grove, Pa.). Nuclei of the cells will be stained with the DNA dye 4′,6-diamidino-2-phenylindole (DAPI; Molecular Probes, Carlsbad, Calif.) for 5 minutes at 23° C. A widefield Olympus IX70 epifluorescence microscope equipped with standard fluorescein/rhodamine/DAPI/Cy5 filter sets will be used to acquire images with the help of MetaMorph image analysis software (Universal Imaging, Sunnyvale, Calif.). Viability of the nicotine-adenovirus conjugates will be recorded.

While the experiments described herein utilize three nicotine analogs (i.e., CNA, CNI, and trans-S′-hydroxymethylnicotine), it should be understood that any nicotine analog may be adapted for use in the methods described herein.

To test the immune response to nicotine-conjugated adenovirus, the nicotine-conjugates described above will be utilized to immunize Balb/c mice according to the regimen described in Table 1.

TABLE 1
Nicotine vaccine dosing¹

| | | | Hapten |
|---|---|---|---|
| | Hapten labeling | | copies |
| Carrier | density | Carrier dose | per dose |
| KLH | TBD² | TBD² or 40 μg | 2.5 × 10¹² or |
| | | | TBD² |
| DNP-ovalbumin- | TBD² | TBD² or 40 μg of | 2.5 × 10¹² or |
| polystyrene beads | | DNP-OVA | TBD² |
| Adenovirus (viable) | 250 per capsid³ | 10¹⁰ pu | 2.5 × 10¹² or |
| | | | 2.5 × 10¹³ |
| Adenovirus (over- | 1250 per capsid⁴ | 2 × 10⁹ pu | 2.5 × 10¹² or |
| conjugated) | | | 2.5 × 10¹³ |

¹Two dosing strategies are used, all vaccines will be tested as an equal number of hapten copies (2.5 × 10¹² copies); non-adenovirus vaccines will also be tested as doses comparable to published reports; adenovirus vaccines will be tested at a second dose (10¹³ particles); actual dosing will depend on actual hapten labeling density determined at the time of the experiment
²This value will be determined empirically; labeling density will be targeted to correspond to that of adenovirus such that doses will have comparable numbers of particles
³Actual range of hapten labeling density tolerated by adenovirus capsids is approximately 80 to 500
⁴Concentrations of hapten in excess of 500 conjugates per capsid render capsids unable to infect target cells

One adenoviral vector based on human serotype 5 (i.e., Ad5RGD) and one adenoviral vector based upon the chimpanzee serotype 7 (i.e., AdC7RGD) will be utilized as adenoviral carriers since these RGD-modified vectors have been demonstrated to elicit an immune response (Worgall et al., J. Virol., 78: 2572-2580 (2004)). The use of a chimpanzee serotype adenovirus allows for evaluation of the impact of pre-existing immunity to the adenovirus capsid upon readministration of the hapten-carrier complex.

The results of this example confirm the preparation of adenovirus-nicotine conjugates.

### Example 4

This example describes a method of inducing an immune response in vitro and in vivo using adenovirus-nicotine conjugates.

To assess B cell activation in vitro, B cells will be purified from Balb/c mice and then exposed to each of the nicotine conjugates described in Example 3. The nicotine-specific antibody responses will be evaluated in the presence of activated syngeneic T-helper cells with or without syngeneic DC. To investigate if the hapten-carrier combinations influence MHC-dependent T helper response, and ultimately B cell responses, syngeneic DC treated with nicotine-conjugates will be analyzed for their potency in promoting T Helper response toward Type 1 helper T cell (Th1) or Type 2 helper T cell (Th2) and to induce nicotine-specific antibodies in B cells. To evaluate if the immune response against nicotine is dependent on direct exposure to the hapten-carrier complex or whether the response requires interaction with MHC molecules, purified murine B cells from Balb/c mice will be exposed to the nicotine-conjugates at a range of doses. The antigen-exposed B cells will be cultured with syngeneic ConA-activated CD4 helper T cells to induce IgG isotypes. Antibody production will be measured by ELISA against nicotine-BSA after 7-12 days.

Antibody production will be measured by ELISA against nicotine-BSA after 7-12 days. To assess titers of anti-nicotine antibodies, microtiter plates will be coated with a nicotine-bovine serum albumin conjugate at 0.4 μg/well in 0.05 M carbonate-bicarbonate buffer, pH 9.6, and incubated at 4° C. for 12 hours. The plates will be washed three times with PBS and blocked with 5% fat-free milk in PBS. After three washes with PBS+0.05% Tween 20 (PBST), the cell culture medium will be added in sequential two-fold dilutions and incubated for 1 hour. After three washes with PBST, anti-mouse IgG or IgM labeled with horseradish peroxidase will be added for a 1 hour incubation. Detection will be accomplished with a peroxidase substrate kit, and absorbance will be determined at 415 nm. For titer determination, the absorbance values of all dilutions will be extrapolated to the two-fold background value with a linear fit function (Plikaytis et al., J. Clin. Microbiol., 29: 1439-1446 (1991) and Worgall et al., J. Clin. Invest., 115: 1281-1289 (2005)).

To investigate the relative ability of nicotine conjugates to influence MHC-dependent T helper and ultimately B cell responses, syngeneic DCs will be pulsed with the nicotine conjugates on an equal nicotine basis or on an equal molar basis of carrier, followed by assessment of their potency in promoting T helper response toward Th1 or Th2 and to induce nicotine-specific antibodies in B cells. Pulsed bone marrow derived dendritic cells will be co-cultured for 7-12 days with either syngeneic naive T cells or B cells. Anti-nicotine specific Th1 and Th2 response will be addressed by re-stimulating the T cells with dendritic cells pulsed with the nicotine conjugate. INF-γ (Th1) and IL-4 (Th2) response will be evaluated by ELISPOT assay after 24 hours and 48 hours as previously described (Krause et al., J. Virol., 80: 5523-5530 (2006)).

Balb/c mice will be immunized intramuscularly with nicotine-conjugates either with equal amounts of nicotine or with amounts comparable to prior reports (see Table 1). The frequency of nicotine-specific CD4 T lymphocytes will be determined with a gamma interferon (IFN-γ)- and IL-4-specific enzyme-linked immunospot (ELISPOT) assay (R&D Systems, Minneapolis, Minn.) 10 days following immunization. Spleen CD4 T cells will be purified by negative depletion with SpinSep T-cell subset purification kits (StemCell Technologies, Vancouver, BC, Canada). Using this method, the purity of the T cells is typically >95%. Splenic DC will be purified from syngeneic naive animals for use as antigen-presenting cells by positive selection with CD11c MACS beads (Miltenyi Biotec, Bergisch Gladbach, Germany) and double purification over two consecutive MACS LS columns (Miltenyi Biotec). The purity of the D is typically >90%. D (5×10⁶/mL) will be incubated for 3 hours with purified nicotine-BSA protein (100 μg/mL) in RPMI medium supplemented with 2% fetal bovine serum, 10 mM HEPES (pH 7.5), and 10⁻⁵ M β-mercaptoethanol. CD4+T cells (2×10⁵) will be incubated with splenic D with or without nicotine-BSA at a ratio of 4:1 in IL-4 and IFN-γ plates for 48 hours. Following washing, biotinylated anti-IFN-γ or anti-IL-4 detection antibodies will be added, and the plates will be incubated overnight at 4° C. The plates will be washed, and the streptavidin-alkaline phosphatase conjugate will be added. For final spot detection, the 3-amino-9-ethylcarbazole substrate will be applied for 1 hour of incubation and rinsed with H₂O. The spots will be objectively counted by computer-assisted ELISPOT image analysis (Zellnet Consulting, New York, N.Y.). In addition to, or as an alternative to, the ELISPOT assay, intracellular cytokine staining can be performed and assessed by flow cytometry as previously described (Worgall et al., J. Clin. Invest., 115: 1281-1289 (2005)).

To evaluate the humoral immune response to the nicotine-conjugates of Example 3, Balb/c mice will be immunized intramuscularly with the conjugates either at an equal nicotine dose of 2.5×10¹² molecules per injection, or at doses comparable to prior reports (see Table 1). Mice injected with unconjugated carriers will be used as negative controls. Serum will be collected from the tail vein 28 days following immunization. Anti-nicotine-specific total IgM and IgG antibodies will be determined by ELISA as described above, except that mouse serum is used as the test substance rather than cell culture medium.

To examine whether re-administration of the hapten-adenovirus vaccine affects the vaccine efficacy, mice will be immunized with 10¹⁰ particles of either nicotine-Ad5RGD or nicotine-AdC7RGD by intramuscular injection. Four weeks later, 0.1 mL sera will be collected from the mice by retro-orbital puncture, and the mice will receive a second vaccination containing either the same adenovirus-nicotine conjugate or the other serotype. After an additional 28 days, sera will be collected and the titer of anti-nicotine, anti-adenovirus serotype 5, and anti-adenovirus serotype C7 antibodies will be assessed. Controls will include single vaccinations and carrier-only vaccinations, both as initial or second vaccinations.

To determine the anti-nicotine titer, CNA-BSA, CNI-BSA, NIC-BSA, or a KLH control (0.1 mL, 0.1 μg/mL) will be added to 96-well microtiter plates and allowed to stand for two hours at room temperature as previously described (Meijler et al., J. Am. Chem. Soc., 125: 7164-7165 (2003)). The plates will be washed with PBS followed by incubation with a solution of powdered milk (1% w/v in PBS, 0.1 mL) for blocking non-specific protein binding. After two hours at 23° C., the plates will be washed with PBS, and serial dilutions of the mouse serum will be added (50 μL per well) and allowed to stand overnight at 4° C. The plates will be washed with PBS, and anti-nicotine antibodies will be detected using a goat anti-mouse horseradish peroxidase secondary antibody (0.01 μg, 50 μL, 37° C., 2 hours). The plates will be washed and treated with substrate solution (50 μL) 3,3′,5,5′-tetramethylbenzidine (0.1 mg in 10 mL of 0.1 M sodium acetate, pH 6.0 with hydrogen peroxide (0.01% w/v)). The plates will be developed in the dark for 30 minutes. Sulfuric acid (1.0 M, 50 μL) will be added to quench the reaction, and the optical density (OD) will be measured at 450 nm. The titer will be determined by the serum dilution that corresponds to 50% of the maximum OD.

To determine antibody specificity, a competition ELISA (Meijler et al., J. Am. Chem. Soc., 125: 7164-7165 (2003)) will be performed using NIC-BSA as the plate antigen with a two-way matrix with dilutions of serum and dilutions of competing agent (nicotine, cotinine, acetylcholine, or N-methylpyrrolidine). The protocol is designed to determine the concentration of the competing agent that reduces serum affinity to the plate-bound antigen by 50%.

To determine antibody affinity, equilibrium dialysis will be performed as previously described (Meijler et al., J. Am. Chem. Soc., 125: 7164-7165 (2003)). Briefly, sera from mice immunized as described above will be diluted 1:100 in PBS and added to 10 wells in a 96-well microtiter plate (60 μL/well). An additional 10 wells per sample in a second microtiter plate will be filled with ³H-nicotine serially diluted in PBS (60 μL total volume). The two plates will be tightly apposed with filled wells facing each other but separated with a dialysis membrane (cutoff 6,000-8,000 Da). The plates will be attached vertically to a shaker and agitated for 6-10 hours at 4° C. After separating the plates, 50 μL will be recovered from each well and transferred to a scintillation vial containing 5 mL of scintillation fluid. The samples will be counted for 5 minutes. The experiment will be repeated twice for each serum sample. The average in differences in DPM between partnered wells will be determined for each concentration of ³H-nicotine, and the K_{d}-avg value will be calculated.

To compare the efficacy of the nicotine-conjugate vaccines, in vivo efficacy will be assessed in mice and rats. The two parameters that will be assessed include biodistribution of nicotine (by assessment of ³H-nicotine in serum and brain) and locomotor activity (assessed by image analysis of digital video recordings). For mouse studies, the same groups/doses will be used as described for single administration experiments in Table 3. Separate groups of mice will be used for biodistribution and locomotor experiments.

For biodistribution studies, the inhibition of nicotine entry into brain will be analyzed in anesthetized mice by intravenous tail vein injection of 0.035 mg/kg unlabeled (−)-nicotine (700 ng in a 20 g mouse) spiked with 3 μCi ³H-labeled (−)-nicotine (#NET827, Perkin Elmer, Waltham, Mass.) (approximately 7 ng based on a specific activity of 70 Ci/mmol). After 3 minutes, the mice will be sacrificed by decapitation and the trunk blood and brain will be collected for analysis of ³H-nicotine content by scintillation counting (Hieda et al., Psychopharmacology (Berl.), 143: 150-157 (1999) and Maurer et al., Eur. J. Immunol., 35: 2031-2040 (2005)). Total nicotine concentration will be extrapolated from radioisotope content and the data will be presented as percent reduction of nicotine uptake in brain/retention in serum relative to the nicotine concentrations found in mice vaccinated with carrier only. Since the majority of the studies on nicotine biodistribution have been performed in rats, a similar study will be duplicated in male Holtzman rats (˜250 g).

Locomotor activity will be determined based on image analysis of digital video collected from an observation chamber, similar to an assay described previously (Pentel et al., Pharmacol. Biochem. Behay., 65: 191-198 (2000)). Mice or rats will undergo a habituation routine which will include placement in the apparatus for 20 minutes, removal from the apparatus for a sham injection (PBS), and return to the apparatus for another 20 minutes. Digital video of the final 5 minutes period will be recorded. Then, the mouse/rat will be removed from the apparatus again, injected with one of three doses of nicotine (0.03, 0.1, or 0.3 mg/kg nicotine), and returned to the apparatus for 20 minutes. Again, the last 5 minutes will be digitally recorded. Three 30-second segments from each observation period will be rendered into individual still images (30 frames/second=900 frames per observation). The frames will be re-assembled as a “stack” using MetaMorph image analysis software (Molecular Devices/MDS, Sunnyvale, Calif.). For white mice/rats, the stack will be collapsed using the “brightest pixel” function such that any pixel that the animal passes through during the 30 sec observation period will become white. The resulting image will map the area of the chamber covered by the mouse/rat during the observation period. A threshold will be applied to the image allowing the white areas to be identified and measured. The percentage of the total apparatus area covered in the three samples from each observation period will be taken as the locomotor score for the period. The ratio of locomotor scores between the injection and sham control analyses will be taken as an indication of the value for the animal.

If the locomotor activity assay is not sensitive enough (e.g., if the activity of nicotine-treated and untreated mice and rats does not differ with a high degree of significance), then self administration assay will be employed (Stewart, J. Psychiatry Neurosci. 25: 125-136 (2000)).

The results of this example will confirm that adenovirus-nicotine conjugates induce immune responses in vitro and in vivo.

### Example 5

This example describes the generation of adenoviral vectors comprising a transgene encoding a protein that stimulates immune cells and a nicotine antigen conjugated to the adenovirus capsid, and the immunogenicity thereof.

To focus the cytokine expression on the dendritic cells that are responding to a hapten, haptens will be conjugated directly to adenovirus vectors that express immunomodulatory genes, and the resulting conjugate will be delivered to dendritic cells ex vivo, with re-administration to mice to evaluate functional consequences. Ex vivo pulsing of dendritic cells with subsequent in vivo analysis has been described for non-hapten targets (Kikuchi et al., Blood, 96: 91-99 (2000) and Worgall et al., Infect. Immun., 69: 4521-4527 (2001)). Three cytokines have been selected for evaluation in this example: one cytokine designed to enhance dendritic cell function (CD40L) (Kikuchi et al., Blood, 96: 91-99 (2000)), one cytokine to enhance B cell function (BAFF) (Tertilt et al., Mol. Ther., 9: 5210 (2004)), and one cytokine to enhance T cell function (IL-15) (Waldmann, Nat. Rev. Immunol., 6: 595-601 (2006)).

To prepare these adenoviral vectors, nicotine will be conjugated to Ad5CD40L and Ad5BAFF, which have been previously described (Kikuchi et al., Blood, 96: 91-99 (2000) and Tertilt et al., Mol. Ther., 9: 5210 (2004)), to AdIL-15, which has been previously described (Ninalga et al., J. Immunother., 28: 20-27 (2005)), and to an adenoviral vector lacking a transgene (AdNull). If the AdIL-15 vector is not comparable to the adenovirus serotype 5 vectors being used in this example, then an Ad51L-15 vector will be constructed using standard molecular biology techniques. As an alternative to nicotine, DNP may be used.

To assess the antibody generating capability of these adenoviral vectors, murine bone marrow derived dendritic cells will be generated from bone marrow precursors. Bone marrow cells harvested from BALB/c mice will be grown in the presence of 10 ng/mL recombinant mouse granulocyte-macrophage colony-stimulating factor and 2 ng/mL recombinant mouse IL-4 (R&D Systems), and used after culture for 7 days. Forty to eighty percent of the non-adherent cells are usually D, characterized by CD83+, CD11c+, CD80+, and CD14−, which has been determined by FACS. D will be further characterized by morphology using Giemsa stain of cytocentrifuge samples, which has revealed the dendritic morphology, and by indirect immunofluorescence for MHC Class II.

The DCs purified from bone marrow, cultured at 5×10⁶ cells/mL, will be transduced for 4 hours with nicotine-AdCD40L, nicotine-AdBAFF, nicotine-AdIL15, or nicotine-AdNull (5000 particles per cell). Naive dendritic cells or dendritic cells infected with unconjugated AdNull will serve as a control. Balb/c mice will receive intravenous tail vein administration of nicotine-adenovirus-modified dendritic cells (5×10⁴ cells in 100 μL PBS). After 28 days, antibodies against nicotine will be assessed in serum by ELISA, competition ELISA, and equilibrium dialysis as described in Example 4.

It is possible that a significant anti-nicotine immune response will only be obtained using RGD-modified adenovirus serotype 5 capsids. Since the immunomodulatory genes are not currently expressed in RGD-modified capsids, the vectors can be engineered by cloning the CD40L, BAFF, or IL15 gene into the Ad5RGD backbone using standard molecular biology techniques.

The results of this example will confirm the preparation of adenoviral vectors comprising a transgene encoding a protein that stimulates immune cells and a nicotine antigen conjugated to the adenovirus capsid, as well as the immunogenicity of the adenoviral vectors.

### Example 6

This example demonstrates a method of inducing humoral immunity in a mammal with adenoviral vectors comprising a cocaine analog conjugated to the adenoviral capsid.

The cocaine analog, GNC, was generated, in brief, by treatment of commercially available (−)-cocaine hydrochloride under acidic conditions, which lead to a double ester hydrolysis to yield an ecgonine core with the correct stereochemistry. A benzyl ester linker was coupled onto the carboxylic acid followed by benzoylation of the secondary alcohol, which yielded the protected hapten. Ester deprotection was performed under a hydrogen atmosphere to yield the GNC product.

GNC was conjugated to the Ad5 (E1/E3-deficient) virion surface (Ad5GNC) at a ratio of 100 GNC molecules per Ad5 capsomere (1:100). Conjugation to Ad was achieved via preactivation of the GNC hapten with 1-ethyl-3-(3-dimethylaminopropyl) carbodiimide (EDC) and sulfo-N-hydroxysuccinimide (S-NHS), followed by addition of the Ad. Attack by protein lysine residues yielded the Ad5GNC conjugate. Compared to mice immunized with an unconjugated control vector, immunization of mice with Ad5GNC elicited cocaine-specific antibody responses, which was demonstrated by the detection of GNC antigen associated with the adenovirus hexon protein on Western blots.

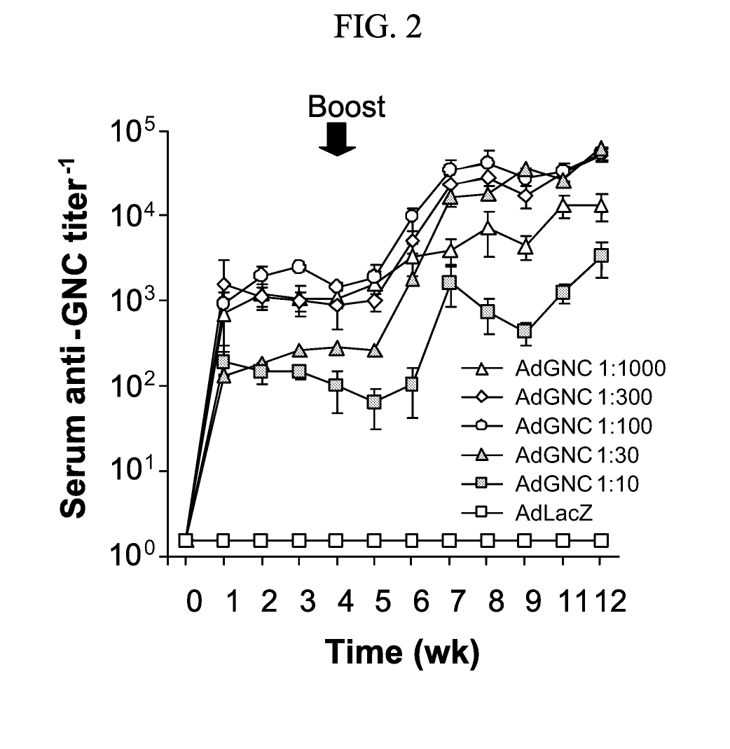

To determine the optimal amount of hapten required to elicit a cocaine-specific antibody response, a variety of conjugates were prepared at various ratios of Ad5 capsomere to GNC molecule (i.e., Ad5:GNC) using S—NHS and EDC chemistry as described herein. Following immunization of mice with these conjugates or a control vector (unconjugated), the timing of serum anti-cocaine antibody development was measured by ELISA. Ratios above 1:30 yielded the highest antibody titers. At 6 weeks post-immunization, the animals were boosted with the homologous Ad5:GNC conjugate. A substantial increase in serum anti-cocaine antibody titers was observed for all conjugates (FIG. 2).

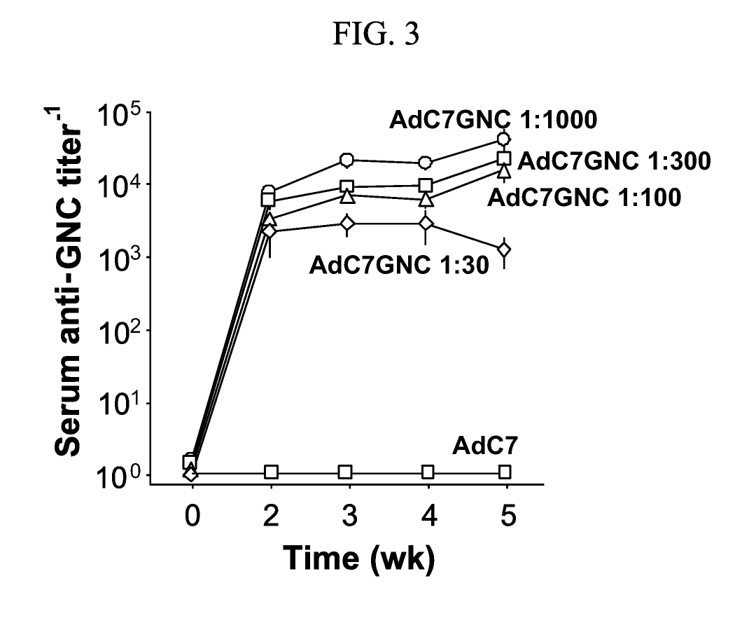

A cocaine vaccine also was developed based on a non-human primate adenovirus (AdC7, E1/E3-deficient), against which there is no pre-existing immunity in the human population. To determine the optimal amount of hapten required to elicit a cocaine-specific antibody response, a variety of conjugates were prepared at various ratios of AdC7 capsomere to GNC molecule (i.e., AdC7:GNC) using Sulfo-NHS and EDC chemistry. Following immunization of mice with these conjugates or a control vector (unconjugated), the timing of serum anti-cocaine antibody development was measured by ELISA. Ratios above 1:30 yielded the highest antibody titers (FIG. 3).

To determine whether the anti-cocaine antibodies alter the pharmacokinetic properties of cocaine and inhibit cocaine from reaching the brain, immunized mice will be challenged with cocaine, and brain and serum will be analyzed for cocaine levels by HPLC.

The experiments described herein can be performed with any antigen of cocaine. For example, GND described previously (Carrera et al., Proc. Nat. Acad. Sci. USA, 98: 1988-1992 (2001); and Carrera et al., Proc. Nat. Acad. Sci. USA, 97: 6202-6206 (2000)) can be used. GNE, which is more stable than GNC, is a novel mono-amide presenting cocaine analog that also can be used. The structures of cocaine and suitable cocaine analogs are as follows:

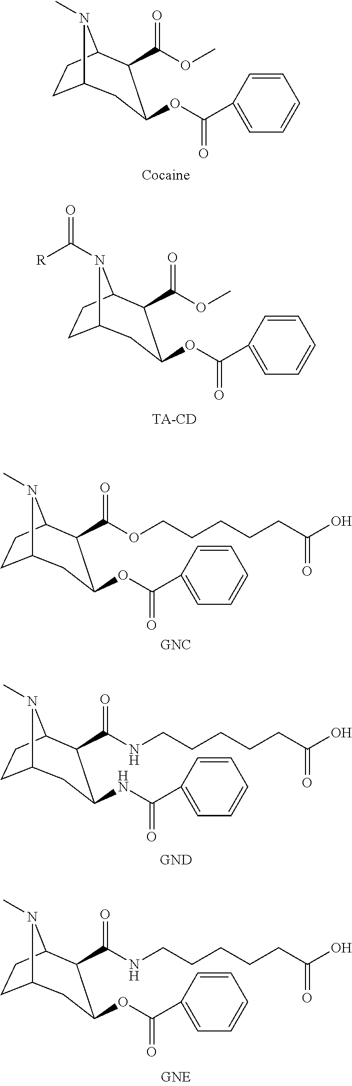

The synthesis of the GNE hapten is similar to GNC. Starting from commercially available (−)-cocaine hydrochloride, saponification of the methyl and benzyl esters under acidic conditions yields an ecgonine core with the desired stereochemistry. Amide coupling of the free acid and an amine linker using EDC activation followed by benzyl esterification of the free alcohol generates the penultimate hapten. Finally, treatment with palladium on carbon under a hydrogen atmosphere deprotects the acid, and prepares the GNE hapten for conjugation to Ad. A scheme of the GNE synthesis is as follows:

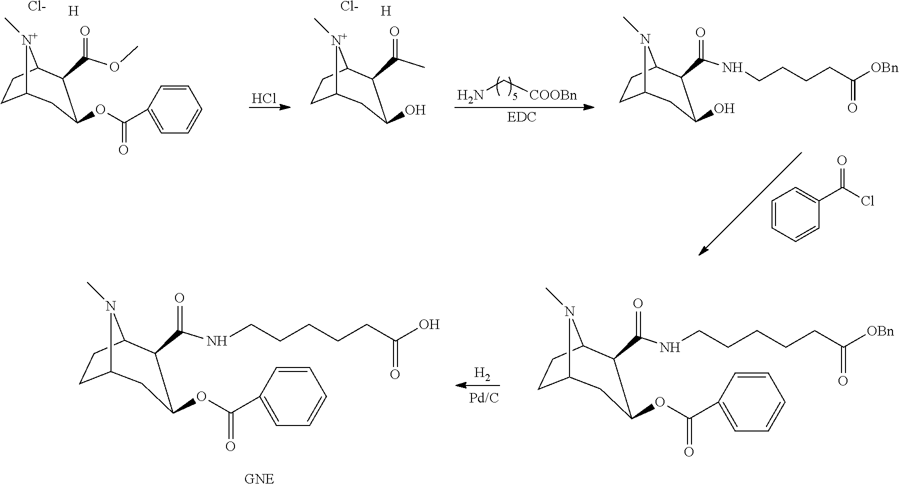

The results of this example demonstrate that conjugation of a cocaine analog to a human or non-human adenovirus can be used to induce anti-cocaine humoral immunity in a mammal.

### Example 7

This example describes rodent models of locomotor activity and drug self-administration that can be used to assess the effects of an adenovirus conjugated to an antigen of cocaine (i.e., Ad-cocaine).

To test the effects of active immunization with the Ad-cocaine vaccines on the psychostimulant activity of cocaine in rats, animals will be immunized with Ad-cocaine or, as negative controls, unconjugated vectors; naive rats will be additional controls. One week after the last booster, the rats will be allowed to habituate in photocell cages overnight. The next day, the rats will receive a saline injection and locomotor activity will be measured for 90 minutes, after which the rats will receive cocaine, and locomotor activity will be measured for another 90 minutes. The rats will receive cocaine for an additional five days and will be tested for locomotor activity after receiving the last dose. One week after the final cocaine treatment, the rats will be challenged with cocaine, and locomotor activities recorded. Rats will receive four additional cocaine challenges, separated by one week, and locomotor activity measured. In addition to measuring locomotor activities, stereotype behaviors will be measured at each experimental time point. The dose of cocaine will be chosen based on previous studies (Carrera et al., Nature 379: 727-730 (1995)).

To test the effects of Ad-cocaine vaccination on cocaine self-administration, extinction and reinstatement of responding for cocaine in rats, Wistar rats will be catheterized with indwelling catheters. One week post-surgery, the rats will be trained to self-administer cocaine for one hour under a fixed-ratio (FR) schedule at least for one week and allowed to self-administer cocaine under alternating FR and progressive-ratio (PR) schedules for another week (baseline period). Then, the rats will be divided into two groups balanced by the number of injections per session during the last 2 FR and PR sessions. One group will be immunized with the vaccines while the other group will be injected with Ad without a hapten. Two weeks after the first immunization, the rats will be allowed to self-administer cocaine for two weeks under FR and PR schedules (self-administration test 1), after which the rats will receive the 2nd immunization or injection with Ad without a hapten. One week after the 2nd immunization, the rats will self-administer cocaine for two weeks (self-administration test 2) then will receive the 3rd immunization. One week after the 3^{rd} immunization, the rats will be tested for cocaine self-administration under FR and PR schedules for two more weeks (self-administration test 3), then will go through extinction sessions where all the conditions are the same as the cocaine self-administration session except that a lever press has no consequence (no cocaine delivery). Extinction sessions will last for a minimum of 10 days and until responding decreases less than 25% of cocaine self-administration. After extinction of responding, the rats will receive cocaine immediately before a reinstatement session, and responding for cocaine will be measured under the same condition as the extinction session. Over the course of the experiment, blood samples will be collected for the determination of the blood antibody levels/affinity levels: three weeks after the 1st immunization, one week after the 2nd immunization, and after the reinstatement session.

To assess cocaine self-administration, rats will be immunized with a 1st intramuscular immunization, four weeks after which they will receive the 2nd booster injections. Depending on the titer, and the initial studies in rats, a 3rd booster may be given at three weeks after the 2nd vaccine administration. Doses will be determined by the initial studies. A standard operant chamber that is placed in a light- and sound-attenuating cubicle will be used for intravenous self-administration. The start of a session will be signaled by the extension of two response levers into the chamber. Responding on the right lever will result in the delivery of a drug injection over 4 seconds. Pressing the left lever will be counted but will have no other programmed consequences. Wistar rats will be implanted with a silastic catheter into the right external jugular vein. Rats will be trained daily to self-administer 0.5 mg/kg/injection of cocaine under an FR1 schedule of reinforcement where one lever press results in the delivery of drug. The data will be expressed as the mean number of injections per session and mean mg/kg per session for each group of rats. Daily cocaine self-administration will be compared between experimental and control groups using a 2-way repeated-measures ANOVA followed by the Bonferroni post hoc test (group×daily session). For extinction and reinstatement, responding per session will be compared between groups using a two-way repeated-measures ANOVA followed by the Bonferroni post hoc test (group daily session). Locomotor activity will be compared using a two-way repeated-measures ANOVA followed by Bonferroni post hoc tests (group treatment day).

The clinical development of a conjugated Ad-based vaccine for cocaine addiction is dependent on the development of a FDA-complaint manufacturing method and a satisfactory toxicology profile. The optimal vector production method will be transitioned to the Weill Cornell Good Manufacturing Practice (GMP) Facility using a GMP-compliant protocol by investigation of conjugation parameters and chemistry on a systematic basis including making multiple batches under various conditions to identify critical control parameters. Validated assays will be developed and specifications established to characterize the product including assessment of conjugation level, residual gene transfer activity, aggregation, stability, batch reproducibility, residuals of conjugation intermediates and potency. With the GMP-produced vaccine, a toxicology study will be performed in rats to establish dose ranges and to identify target organs for caution in further development. This will include n=8 rats/group (4 male, 4 female) with injection of 3 doses plus PBS control with assessment for survival and overt side effects. Rats will be sacrificed for at 3, 7, 30, and 180 days for serum chemistry, complete blood count, organs weights, and histopathological assessment of 13 organs (2 slides per organ) by a certified veterinary pathologist.

The results of this example will confirm that vaccination of rats with adenovirus-cocaine conjugates induces changes in locomotor activity in response to a cocaine challenge, and affects cocaine self-administration.

### Example 8

This example demonstrates a method of inducing an immune in a non-human primate using adenovirus-cocaine conjugates.

Three assessments will be used as measures of efficacy: (1) decreases in cocaine self-administration; (2) decreases in pharmacokinetics of cocaine and its metabolites; and (3) decreases in the ability of cocaine to bind to the dopamine transporter. For each parameter, baseline dependent measures will be obtained before vaccine administration and will be assessed repeatedly after vaccine administration over a period of 12 months.

After a three month quarantine period and a three month training period, monkeys will have vascular access port surgery and will be subsequently trained to self-administer intravenous (i.v.) cocaine. At baseline, monkeys will respond for cocaine and candy until behavior is stable. Two cocaine pharmacokinetic sessions will be conducted to determine the pharmacokinetics of i.v. cocaine and its metabolites. All monkeys will have a baseline MRI and PET scans in month 8. At the end of month 9, four monkeys will be given an intramuscular injection of the vaccine followed in one month by a boost. The doses will be determined from the mice and rat studies, and likely will be 10¹¹ to 10¹² particle units. The other two monkeys will be given vehicle as a control. Over the next 12 months, the vaccine efficacy will be determined by repeatedly measuring changes in cocaine self-administration. Monkeys will have self-administration choice sessions between i.v. cocaine and candy five days per week. The pharmacokinetics of cocaine will be reassessed each month. PET scans will be repeated every other month and antibody titers will be measured weekly for the first six weeks and then monthly thereafter. In the event the vaccine is effective, monkeys will be sacrificed so that toxicology can be performed.

The ability of Ad-cocaine to alter the pharmacokinetics of cocaine will be relevant to any concurrent changes in cocaine self-administration, since similar studies have been performed following acute and repeated cocaine administration in rhesus monkeys (Evans and Foltin, Neuropsychopharmacology, 29: 1889-1900 (2004) and Pharmacol. Biochem. Behay., 83: 56-66 (2006)). In these studies, the effects of a range of cocaine doses were assessed in female rhesus monkeys during four phases of the menstrual cycle. Herein, cocaine pharmacokinetics will be conducted at baseline (months 8 and 9 before vaccine administration), and on a monthly basis after vaccine administration. Monkeys will receive four doses of cocaine separated by 15 minutes. Cocaine and cocaine metabolite levels will be measured five minutes after each cocaine dose and five to 120 minutes after the last cocaine dose.

A non-human primate model of intravenous cocaine self-administration will be used to determine if vaccination with Ad-cocaine decreases drug taking, which will likely provide the best predictor of clinical efficacy. To control for nonselective effects, monkeys will be trained to self-administer cocaine and candy, using a choice procedure. If the vaccine is effective at blocking the effects of cocaine, monkeys should decrease the number of cocaine choices self-administered and correspondingly increase the number of candy choices. A non-specific effect would be evidenced by a decrease in both cocaine and candy choices. After all monkeys have been trained to enter the primate chair and respond on the levers for candy, they will have vascular access port surgery (VAP). For self-administration sessions, monkeys will be placed into custom-built primate chairs, moved to the workstation, and connected to the infusion apparatus. Sessions will be conducted five days per week. Monkeys will initially be trained to self-administer 0.1 mg/kg cocaine per infusion. Once responding is stable, the cocaine dose available will be 0.05 mg/kg/infusion. They will respond on one lever for 0.05 mg/kg/infusion cocaine on a fixed ratio 50 schedule and they will respond on another lever for candy on a fixed ratio 10 schedule of reinforcement. The primary dependent measure will be the number of cocaine doses self-administered each session. Choice data after the vaccine will be compared to the 95% confidence interval of baseline choice data for each monkey and to antibody levels.

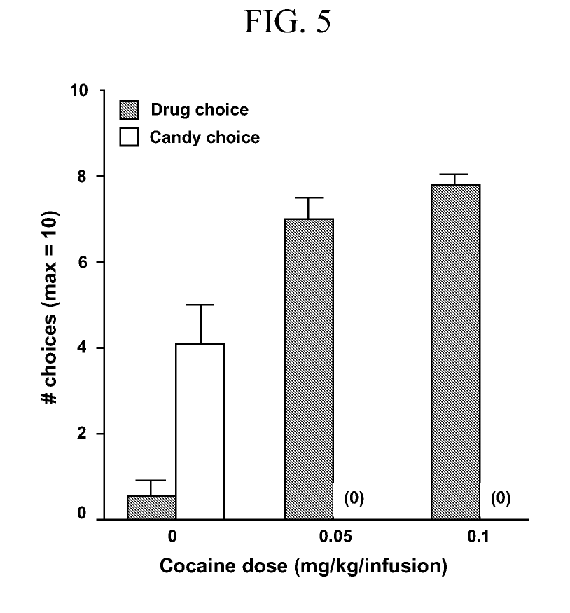

A study was performed of a representative monkey (among a group of 4 female monkeys) which self-administered i.v. cocaine or candy using a schedule of reinforcement similar to the aforementioned schedule. When placebo cocaine was available, the monkey chose candy over cocaine, but when either dose of cocaine was available, the monkey exclusively chose cocaine over candy (FIG. 5).

To image the dopamine transporters (DAT) that bind to cocaine, positron emission tomography (PET) radioligand imaging will be performed, which will allow for the direct measurement of the ability of the vaccine to prevent cocaine from entering the brain. In rhesus monkeys, doses of 0.1 and 1.0 mg/kg cocaine occupy 53% and 87% of the DAT (Votaw et al., Synapse, 44: 203-210 (2002)). In human cocaine abusers, at least 47% of the DAT needs to be occupied in order to perceive the subjective effects of cocaine (Volkow et al., Nature, 386: 827-830 (1997)). The six rhesus monkeys will undergo PET scans with the radiotracer [11C]PE2I, which labels the DAT. Prior to vaccination, the monkeys will be scanned with [11C]PE2I using the dose of cocaine that occupies 87% of the DAT (1.0 mg/kg). The monkeys will be rescanned every two months following vaccination, using the same dose of cocaine. Each monkey will undergo PET scans in the following order: (1) pre-vaccine, a baseline (no cocaine) scan followed by an occupancy scan (following 1.0 mg/kg i.v. cocaine), and (2) post-vaccine, occupancy scans (following 1.0 mg/kg iv cocaine) at months 10, 12, 14, 16, 18, and 20. All monkeys will undergo an MRI for delineation of the regions of interest (primarily striatum). The cerebellum, which is devoid of DAT binding, will be used as the reference region to measure free and nonspecifically bound radiotracer. A kinetic analysis will be performed on all scans, using a 1-tissue compartment model (1TC) for the cerebellum and a 2-tissue compartment model (2TC) for the striatum. The outcome measure will be binding potential, defined as the ratio of specific binding to the arterial plasma. In order to measure DAT occupancy, intravenous cocaine (1.0 mg/kg) will be administered just prior to the radiotracer. The percent change in BP obtained from the baseline (no cocaine) scan and that obtained following the dose of cocaine provides the percent occupancy of cocaine at the DAT. The percent occupancy will be calculated prior to and following vaccination. The statistical analysis will consist of the comparison of % occupancy before and after vaccination in each animal, two-tailed t-test for within-subject comparison.

All vaccinations in non-human primates will be carried out using an adenovirus-cocaine conjugate produced in a GMP facility so that the data can be used in support of a future IND for a clinical study.

The results of the example will confirm that vaccination of non-human primates with adenovirus-cocaine conjugates induces changes in (a) cocaine self-administration, (b) the pharmacokinetics of cocaine and its metabolites, and (c) the ability of cocaine to bind to the dopamine transporter.

### Example 9

This example demonstrates that the adenoviral hexon protein can be modified to increase primary amines for hapten conjugation.

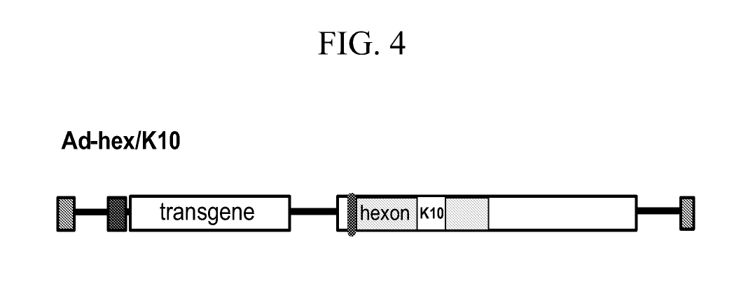

In order to further enhance the immunogenicity of Ad-based addictive drug vaccines, strategies were devised to increase the amount of hapten that can be conjugated onto the Ad virion surface. Exposed lysine residues provide an amine group that is a target for conjugation to the carboxylate group on the hapten. Therefore, increasing the number of lysine residues on the virion capsid should provide more targets for hapten conjugation. It is possible to modify the flexible loops present on the adenovirus hexon protein to incorporate additional amino acid residues without disrupting the virion architecture. An adenovirus containing an insertion within the hypervariable regions of the hexon coding sequence of either 5 or 10 lysine residues was constructed as a platform for “hyper-haptenation” of the virion using standard molecular biology techniques (FIG. 4).

This example describes modifications to the adenoviral hexon protein which increase the number of primary amines for hapten conjugation.

### Example 10

This example describes the generation of an adenovirus conjugated to dinitrophenol (DNP).

Studies involving the analysis of adenoviral particles comprising peptide sequences engineered into an adenovirus capsid have been described (Worgall et al., J. Clin. Invest., 115: 1281-1289 (2005) and Krause et al., J. Virol., 80: 5523-5530 (2006)), and the assays described therein will be utilized to prepare an adenovirus conjugated to DNP. These assays include, for example, analyses of immunoglobulin titer and isotype as well as immunological analyses to identify activation of the Th1 and/or Th2 arms of the immune system.

Conjugation of small molecules to adenovirus capsids has extensively made use of succinimidyl ester cross-linking to epsilon amino groups exposed on the capsid surface (Leopold et al., Hum. Gene Ther., 9: 367-378 (1998) and Miyazawa et al., J. Virol., 73: 6056-6065 (1999)). Using the same protocol, DNP will be coupled to the adenovirus capsid and other vaccine carriers for comparison as described in Table 2. DNP is well known in the art as a model hapten (see., e.g., Gell and Benacerraf, J. Exp. Med., 113: 571-585 (1961) and Kantor et al., J. Exp. Med., 117: 55-69 (1963)).

TABLE 2
DNP-conjugate vaccination groups

Hapten-carrier vaccine (soluble protein)
DNP-ovalbumin
Hapten-carrier vaccine (protein aggregate)
DNP-KLH
Nanoparticle vaccines
DNP-KLH linked to polystyrene beads (20, 40, 100, 200 nm diameter)
DNP-adenovirus vaccines, native capsids
DNP-adenovirus serotype 5, empty capsid (DNP-Ad5empty)
DNP-adenovirus serotype 5 (DNP-Ad5)
DNP-adenovirus serotype C7 (DNP-AdC7)
DNP-adenovirus vaccines, broad tropism
DNP-adenovirus serotype 5 with additional integrin binding RGD
sequences genetically encoded in the fiber proteins (DNP-Ad5RGD)
DNP-adenovirus serotype C7 with additional integrin binding RGD
sequences genetically encoded in the fiber proteins (AdC7RGD)
DNP-adenovirus vaccines, tropism ablated
DNP-adenovirus serotype 5 with mutation ablating fiber-CAR interaction
(DNP-Ad5F*)
DNP-adenovirus serotype 5 with mutation ablating penton base-integrin
interaction (DNP-Ad5PB*)
DNP-adenovirus serotype 5 with mutation ablating both the fiber-CAR
interaction and the penton base-integrin interaction (DNP-Ad5F*PB*)

In particular, 1 mg DNP-X succinimidyl ester (Invitrogen, Carlsbad, Calif.) will be reconstituted in 1 mL NaHCO₃ buffer, filtered through a 0.2 μm syringe tip filter, and then added to a suspension of adenovirus for a final capsid concentration of 10¹² particles/ml (concentration determined by absorbance (Mittereder et al., J. Virol., 70: 7498-7509 (1996)) and 0.2 mg/mL DNP. The mixture is maintained at 23° C. for 30 minutes with mixing by inversion every ten minutes. The reaction mixture is transferred to a dialysis cassette (Slide-A-Lyzer, 10,000 MWCO, 0.1-0.5 mL or 0.5-3 mL size depending on reaction volume; Pierce Thermo Fisher Scientific, Rockford, Ill.) and dialyzed overnight against 1000× volumes of dialysis buffer (10 mM Tris, 10 mM MgCl₂, 150 mM NaCl, 10% glycerol, pH to 7.8) at 4° C. with gentle stirring. After one change of dialysis buffer and an additional two hours of dialysis, the viral suspension is harvested from the chamber, combined with 100% glycerol to achieve a 30% final concentration of glycerol as a cryoprotectant, mixed, and stored at −20° C. until use.

The extent of DNP conjugation will be determined by measuring the absorbance due to DNP at 360 nm (ε360=17,400 (Good et al., Selected Methods in Cellular Immunology, pp. 343-350, W.H. Freeman & Co, San Francisco, Calif. (1980)) and applying Beer's Law to determine the molar concentration of DNP. Care will be taken to compare the absorbance curve of adenovirus in the absence and presence of DNP conjugation to determine the deflection from baseline absorbance specifically attributable to DNP. This absorbance will be used in the Beer's Law calculation. The molar concentration of adenovirus is calculated from the particle concentration prior to labeling, using an extinction coefficient of 17,400 for DNP-lysyl at 360 nm. To characterize the sites of protein modification by DNP on adenovirus, a Western blot after SDS-PAGE will be performed as previously described (Vincent et al., J. Virol., 75: 1516-1521 (2001)). Ad capsid proteins (5×10¹⁰ particles/lane) will be denatured for 10 minutes at 95° C. in Laemmli sample buffer containing 6 M urea, separated on a 4 to 20% polyacrylamide gradient gel, transferred to nitrocellulose (Hybond-C, Amersham, Uppsala, Sweden), blocked with 5% dried milk in Tris-buffered saline, pH 7.4, and probed using human anti-adenovirus sera (1:1,000 dilution) or murine anti-DNP antibodies (monoclonal anti-DNP antibody, Sigma-Aldrich, St. Louis, Mo.). Achieving a conjugation rate of 0.3 to 2.0 DNP molecules per capsomere (or approximately 80 to 500 DNP molecules per capsid) would be comparable to conjugation levels observed for the fluorophore, Cy3, as described previously (Leopold et al., Hum. Gene Ther., 9: 367-378 (1998)). Higher conjugation rates are expected to impair the ability of the capsid to deliver the adenovirus genome to the nucleus. Both DNP-conjugated and DNP-“overconjugated” preparations will be developed for testing.

To provide a comprehensive evaluation of adenovirus as a hapten carrier, DNP will be presented as an immunogen using several other carriers (see Table 2). DNP-ovalbumin (DNP-OVA, loading ratio 7, Biosearch Technologies, Novato, Calif.) will be used to reflect immunization by a hapten linked to a soluble foreign protein. DNP-keyhole limpet hemocyanin (DNP-KLH, loading ratio 21, Biosearch Technologies) is a classic combination of hapten and carrier. Of interest, KLH exists as a suspension of protein aggregates with molecular weights ranging from 450,000 to 17 million, easily qualifying as a nanoparticle. In this respect, KLH is similar to VLPs and the adenovirus capsid in size. Data provided by Biosearch Technologies on the extent of protein modification (to be confirmed using 360 nm absorbance as described above) allows dosing to be based on two strategies. Animals will receive either an equal dose of DNP or they will receive doses of vaccine comparable to prior published reports. The dosing regimen with DNP conjugates will be similar to that provided in Table 1 with regard to nicotine dosing.

Precise dosing will be determined at the time the lot release data are received from the vendor (e.g., Biosearch Technologies). As a control to demonstrate the contribution of the hapten and carrier delivered in particulate form, DNP-KLH will be conjugated to carboylate-modified polystyrene beads of varying size (FluoSpheres with yellow/green fluorescent dye, #F8787, 8795, 8803, and 8811, Invitrogen, Carlsbad, Calif.). Coupling will be performed according to the manufacturer's instructions. 10-25 mg of protein (DNP-KLH) will be suspended at 2-5 mg/mL in MES buffer in a glass centrifuge tube. A 5 mL volume of a 2% aqueous suspension of carboxylate-modified microsphere will be added and incubated at 23° C. for 15 minutes. After addition of 40 mg of EDAC (1-ethyl-3-(3-dimethylaminopropyl)carbodiimide, hydrochloride; E2247, Invitrogen, Carlsbad, Calif.) and mixing, the pH will be adjusted to 6.5±0.2 with dilute NaOH. The reaction mixture will be incubated on a rocker or orbital shaker for 2 hours at 23° C. and then quenched by addition of solid glycine to give a final concentration of 100 mM. After an additional 30 minutes at room temperature, the reaction will be dialyzed using 100,000 MW cutoff dialysis cassettes (see above) into 50 mM PBS+1% syngeneic mouse serum to stabilize the particles. Finally, the solution will be combined with 100% sterile glycerol to a final concentration of 30% glycerol and stored at −20° C. until use. DNP concentration of the preparation will be assessed based on absorbance at 360 nm as described above. Absorbance curves (200 nm through 800 nm) of yellow/green fluorescent polystyrene beads coupled with unconjugated proteins will be assessed and compared to absorbance curves with DNP-conjugated protein beads so that the deflection in the baseline absorbance attributable to DNP can be accurately determined. Dosing of beads for immunization reactions will be determined by either equal DNP dose or on doses comparable to prior published reports.

The experiments described in this example can be performed with any hapten. For example, fluorescein is also well known as a hapten, and a variety of fluorescein-modified proteins and anti-fluorescein antibodies are commercially available. The use of fluorescein would also simplify the trafficking studies both in vitro and in vivo. In addition, carboxyfluorescein-conjugated adenovirus has been previously described (Leopold et al., Hum. Gene Ther., 11: 151-165 (2000) and Miyazawa et al., J. Virol., 75: 1387-1400 (2001)). Due to the fact that both DNP-conjugation and EDAC-mediated carboxylate conjugation will target amines on the KLH, it is possible that overconjugation of KLH with DNP will decrease the efficiency of linking to the carboxylate beads. If this is encountered, DNP-KLH with lower loading ratios will be used. If lower loading ratios are not available, then DNP-KLH conjugations will be performed using DNP-X succinimidyl ester (Invitrogen, Carlsbad, Calif.) and purified proteins.

The results of this example will confirm the generation of adenovirus-dinitrophenol (DNP) conjugates.

### Example 11

This example describes the ability of DNP conjugates to induce an immune response in vitro and in vivo.

The experiments described herein will compare the immune stimulatory effects of the DNP-conjugates of Example 10, in order to assess whether the biochemical or physical character of the conjugates correlates with immune response. The DNP-conjugates will be administered to mice either directly or via syngeneic transfer of pulsed dendritic cells (D) to evaluate the relative efficacy of adenovirus capsids as nanoparticle vaccines to induce immunity and antigen presentation.

To assess B cell activation in vitro, B cells will be purified from Balb/c mice, and then exposed to each of the DNP-conjugates of Example 10. The DNP-specific antibody responses and anti-DNP antibody titer determination will be evaluated in the presence of activated syngeneic T-helper cells with or without syngeneic D as described in Example 4.

To investigate the relative ability of DNP-conjugates to influence MHC-dependent T helper and ultimately B cell responses, syngeneic dendritic cells will be pulsed with the DNP conjugates on an equal DNP basis or on an equal molar basis of carrier, followed by assessment of their potency in promoting T Helper response toward Th1 or Th2 and to induce DNP-specific antibodies in B cells by ELISAPOT as described in Example 4.

The humoral immune response to the DNP-conjugates will be performed in Balb/c mice as described in Example 4. The experiments described herein and in the above Examples compare methods of hapten delivery for immunization. As such, the relative efficacy of each carrier should be independent of the MHC haplotype of the mouse strain. The strain described above, Balb/c, is MHC haplotype H-2d, and was chosen to provide a comparison with the experiments described in Example 4 in which Balb/c will also be used. If poor immune responses to DNP are observed in Balb/c mice, then mice with other MHC haplotypes (e.g., Balb/b, H-2b or Balb/k, H-2k) will be tested. Furthermore, the experiments described herein and in Example 4 utilize nanoparticle vaccines without adjuvants. Adjuvants can be effective in boosting immune responses but are empirical in application. Nevertheless, it should be understood that adjuvants can be included in any of the immunization protocols described herein.

The results of this example will confirm the ability of DNP conjugates to induce an immune response in vitro and in vivo.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of inducing an immune response against an addictive drug in a human, which method comprises administering to a human an adenovirus-antigen conjugate comprising an adenovirus with a coat protein and an antigen of an addictive drug conjugated to the coat protein of the adenovirus, whereby the antigen is presented to the immune system of the human to induce an immune response against the addictive drug in the human.

2. The method of claim 1, wherein the antigen is a small molecule.

3. The method of claim 2, wherein the small molecule is a hapten.

4. The method of claim 1, wherein the coat protein comprises at least one non-native lysine residue.

5. The method of claim 4, wherein the coat protein comprises 5 to 10 non-native lysine residues.

6. The method of claim 1, wherein at least one native lysine residue is absent from the coat protein.

7. The method of claim 1, wherein the coat protein is a hexon protein.

8. The method of claim 7, wherein the hexon protein comprises at least one non-native lysine residue in one or more flexible loops of the hexon protein.

9. A method of reducing the effect of an addictive drug in a human, which method comprises administering to a human an adenoviral vector comprising a nucleic acid sequence which encodes an antibody directed against an addictive drug and which is operably linked to a promoter, whereby the nucleic acid sequence is expressed in the human to produce the antibody and reduce the effect of the addictive drug in the human.

10. The method of claim 1, wherein the addictive drug is selected from the group consisting of opioids, morphine derivatives, depressants, dissociative anesthetics, cannabinoids, hallucinogens, stimulants, prescription medications, anabolic steroids, inhalants, and club drugs.

11. The method of claim 1, wherein the addictive drug is selected from the group consisting of cocaine, fentanyl, heroin, morphine, opium, oxycodone, hydrocodone, ketamine, PCP, barbiturates, benzodiazepines, flunitrazepam, GHB, methaqualone, hashish, marijuana, LSD, mescaline, psilocybin, amphetamine, cocaine, MDMA, methamphetamine, methylphenidate, nicotine, and analogs thereof.

12. The method of claim 11, wherein the addictive drug is nicotine or an analog thereof.

13. The method of claim 12, wherein the addictive drug is the nicotine analog AM3.

14. The method of claim 11, wherein the addictive drug is cocaine or an analog thereof.

15. The method of claim 14, wherein the addictive drug is the cocaine analog GNC or GNE.

16. The method of claim 1, wherein the adenovirus or adenoviral vector is replication-deficient.

17. The method of claim 1, wherein the adenovirus or adenoviral vector is a human or non-human primate adenovirus or adenoviral vector.

18. The method of claim 17, wherein the adenovirus or adenoviral vector is a human serotype 5 adenovirus or adenoviral vector.

19. The method of claim 17, wherein the adeonvirus or adenoviral vector is a non-human primate serotype C7 adenovirus or adenoviral vector.

20. The method of claim 1, wherein the adenovirus or adenoviral vector further comprises one or more transgenes encoding a protein that stimulates one or more cells of the immune system.

21. The method of claim 20, wherein the one or more transgenes encode a protein that stimulates B cell activity.

22. The method of claim 20, wherein the transgene encodes B-cell Activating Factor (BAFF).

23. An adenovirus-antigen conjugate comprising (a) an adenovirus with a coat protein and (b) an antigen of an addictive drug conjugated to the coat protein of the adenovirus.

24. The adenovirus-antigen conjugate of claim 23, wherein the antigen is a small molecule.

25. The adenovirus-antigen conjugate of claim 24, wherein the small molecule is a hapten.

26. The adenovirus-antigen conjugate of claim 23, wherein the coat protein comprises at least one non-native lysine residue.

27. The adenovirus-antigen conjugate of claim 26, wherein the coat protein comprises 5 to 10 non-native lysine residues.

28. The adenovirus-antigen conjugate of claim 23, wherein at least one native lysine residue is absent from the coat protein.

29. The adenovirus-antigen conjugate of claim 23, wherein the coat protein is a hexon protein.

30. The adenovirus-antigen conjugate of claim 29, wherein the hexon protein comprises at least one non-native lysine residue in one or more flexible loops of the hexon protein.

31. The adenovirus-antigen conjugate of claim 23, wherein the addictive drug is selected from the group consisting of opioids, morphine derivatives, depressants, dissociative anesthetics, cannabinoids, hallucinogens, stimulants, prescription medications, anabolic steroids, inhalants, and club drugs.

32. The adenovirus-antigen conjugate of claim 23, wherein the addictive drug is selected from the group consisting of cocaine, fentanyl, heroin, morphine, opium, oxycodone, hydrocodone, ketamine, PCP, barbiturates, benzodiazepines, flunitrazepam, GHB, methaqualone, hashish, marijuana, LSD, mescaline, psilocybin, amphetamine, cocaine, MDMA, methamphetamine, methylphenidate, nicotine, and analogs thereof.

33. The adenovirus-antigen conjugate of claim 32, wherein the addictive drug is nicotine or an analog thereof.

34. The adenovirus-antigen conjugate of claim 33, wherein the addictive drug is the nicotine analog AM3.

35. The adenovirus-antigen conjugate of claim 32, wherein the addictive drug is cocaine or an analog thereof.

36. The adenovirus-antigen conjugate of claim 35, wherein the addictive drug is the cocaine analog GNC or GNE.

37. The adenovirus-antigen conjugate of claim 23, wherein the adenovirus is replication-deficient.

38. The adenovirus-antigen conjugate of claim 23, wherein the adenovirus is a human adenovirus or a non-human primate adenovirus.

39. The adenovirus-antigen conjugate of claim 38, wherein the adenovirus is a human serotype 5 adenovirus.

40. The adenovirus-antigen conjugate of claim 38, wherein the adenovirus is a non-human primate serotype C7 adenovirus.

41. The adenovirus-antigen conjugate of claim 23, wherein the adenovirus further comprises one or more transgenes encoding a protein that stimulates one or more cells of the immune system.

42. The adenovirus-antigen conjugate of claim 41, wherein the one or more transgenes encode a protein that stimulates B cell activity.

43. The adenovirus-antigen conjugate of claim 41, wherein the transgene encodes B-cell Activating Factor (BAFF).

44. A composition comprising the adenovirus-antigen conjugate of claim 23 and a carrier therefor.

45. An adenoviral vector comprising a nucleic acid sequence which encodes an antibody directed against an addictive drug and which is operably linked to a promoter, wherein the nucleic acid sequence can be expressed in a human to produce the antibody.

46. The adenoviral vector of claim 45, wherein the addictive drug is selected from the group consisting of opioids, morphine derivatives, depressants, dissociative anesthetics, cannabinoids, hallucinogens, stimulants, prescription medications, anabolic steroids, inhalants, and club drugs.

47. The adenoviral vector of claim 45, wherein the addictive drug is selected from the group consisting of cocaine, fentanyl, heroin, morphine, opium, oxycodone, hydrocodone, ketamine, PCP, barbiturates, benzodiazepines, flunitrazepam, GHB, methaqualone, hashish, marijuana, LSD, mescaline, psilocybin, amphetamine, cocaine, MDMA, methamphetamine, methylphenidate, nicotine, and analogs thereof.

48. A composition comprising the adenoviral vector of claim 45 and a carrier therefor.


SEQUENCE LISTING (3 sequences)

SEQ ID NO: 1 - protein, 5 aa, Artificial sequence
  RGD motif 1
CRGDC

SEQ ID NO: 2 - protein, 9 aa, Artificial sequence
  RGD motif 2
CXCRGDCXC

SEQ ID NO: 3 - protein, 9 aa, Artificial sequence
  RGD motif 3
CDCRGDCFC